(19) United States
(12) Patent Application Publication
(10) Pub. No.: US 2024/0032955 A1
(43) Pub. Date: Feb. 1, 2024

(54) SURGICAL INSTRUMENTS, GUIDES, AND METHODS OF USE

(71) Applicant: Paragon 28, Inc., Englewood, CO (US)

(72) Inventors: Michael SCHMIDT, Greenwood Village, CO (US); Richard David HUNT, Arvada, CO (US); Albert DACOSTA, Lone Pine, CO (US); Joseph DOGUÉ, Aurora, CO (US); Matt JARBOE, Ada, MI (US)

(73) Assignee: Paragon 28, Inc., Englewood, CO (US)

(21) Appl. No.: 18/482,496

(22) Filed: Oct. 6, 2023

Related U.S. Application Data
(63) Continuation of application No. PCT/US22/71638, filed on Apr. 8, 2022.
(60) Provisional application No. 63/173,043, filed on Apr. 9, 2021.
(60) Provisional application No. 63/262,845, filed on Oct. 21, 2021.

(51) Int. Cl. A61B 17/28

(52) U.S. Cl. CPC A61B 17/28; A61B 2017/2808; A61B 2017/00367

(57) ABSTRACT

Disclosed is a clamp that has an actuation portion and an engagement portion coupled with the actuation portion by a coupling portion. The engagement portion includes a first arm having a first engagement element arranged at an end of the first arm, and a second arm having a second engagement element arranged at an end of the second arm. A surgical system is disclosed that includes a clamp with an actuation portion and an engagement portion having a first arm and a second arm. The system includes a cutting guide and a fixation device. A surgical method is disclosed that includes aligning a retention element; releasably coupling a surgical clamp with the retention element; manipulating an actuator; manipulating the surgical clamp; coupling a stabilization wire; reducing an angle between two bones; derotating a bone; inserting a second stabilization wire; making a cut to a bone; and implanting a fusion device.

### CROSS REFERENCE TO RELATED APPLICATIONS

This application is a bypass continuation of PCT Application No. PCT/US2022/071638, filed Apr. 8, 2022, and entitled “Surgical Instruments, Guides, and Methods of Use,” which claims priority benefit under 35 U.S.C. § 119(e) of U.S. Provisional Application No. 63/173,043 filed Apr. 9, 2021, and U.S. Provisional Application No. 63/262,845 filed Oct. 21, 2021, both entitled “Surgical Instruments, Guides, and Methods of Use,” which are both incorporated herein by reference in their entirety.

### TECHNICAL FIELD

The present disclosure relates to surgical instruments, guides, and methods of use to be implemented in surgical procedures. The present disclosure relates to podiatric and orthopedic surgical instruments, guides, and methodology to be implemented in various procedures of the foot and/or ankle, for example arthrodesis. More specifically, but not exclusively, the present disclosure relates to surgical instruments, guides to be implemented in conjunction with instruments (as well as other components, for example implants, devices, systems, assemblies, etc.) and methods of use for performing arthrodesis procedures of the Lapidus joint.

### BACKGROUND OF THE INVENTION

Many currently available surgical instruments and guides, as well as methodology, do not completely address the needs of patients. Additionally, many currently available surgical instruments, guides, and methodology fail to account for properties of joint anatomy and accordingly can decrease favorability of the outcome for the patient.

### SUMMARY OF THE INVENTION

The present disclosure is directed toward surgical guides for implementation in conjunction with implants, instruments, and methods directed to arthroplasty procedures.

A first aspect of the present disclosure is a clamp. The clamp includes an actuation portion and an engagement portion coupled with the actuation portion by a coupling portion. The engagement portion includes a first arm having a first engagement element arranged at a distal end of the first arm and a second arm having a second engagement element arranged at a distal end of the second arm.

According to the first aspect of the present disclosure, the first engagement element is different from the second engagement element.

According to the first aspect of the present disclosure, the second engagement element is integral with the second arm.

According to the first aspect of the present disclosure, the second engagement element includes a tapered geometry in the vertical direction.

According to the first aspect of the present disclosure, the second engagement element includes a flat disposed on a medial surface thereof, wherein the flat includes a textured surface.

According to the first aspect of the present disclosure, the textured surface is configured to contact at least a portion of a first metatarsal such that the second engagement element maintains contact with the first metatarsal.

According to the first aspect of the present disclosure, the first arm includes an aperture positioned at a distal end configured to rotatably couple with the first engagement element.

According to the first aspect of the present disclosure, the first actuator is disposed at an end of the first portion opposite the second portion.

According to the first aspect of the present disclosure, the first engagement element includes a retention portion and an actuator.

According to the first aspect of the present disclosure, the retention portion includes a post, at least a portion of which is configured to be received by the aperture of the first arm so as to facilitate rotatable and releasable coupling between the first engagement element and the first arm.

According to the first aspect of the present disclosure, the actuator includes a central opening configured to receive at least a portion of the post such that the actuator is disposed above the aperture of the first arm and above the retention portion.

According to the first aspect of the present disclosure, the retention portion further includes a first retention element and a second retention element extending from the post.

According to the first aspect of the present disclosure, manipulation of the actuator about a threading disposed on a surface of the retention portion adjusts a distance between the first retention element and the second retention element.

According to the first aspect of the present disclosure, the first retention element and the second retention element each include a textured surface configured to interface with a second metatarsal.

According to the first aspect of the present disclosure, the retention portion is rotatable coupled with the aperture of the first arm such that the retention portion comprises a 360-degree range of motion.

According to the first aspect of the present disclosure, the first arm has a different geometry from the second arm.

According to the first aspect of the present disclosure, the first engagement element has a depth different than that of the second engagement element.

A second aspect of the present disclosure is a surgical system. The surgical system includes a clamp. The clamp includes an actuation portion and an engagement portion. The engagement portion is coupled with the actuation portion by a coupling portion disposed between the engagement portion and the actuation portion. The engagement portion includes a first arm with a first engagement element arranged at a distal end of the first arm, wherein the first engagement element is integral with the first arm and configured to contact a surface of a first bone. The engagement portion also includes a second arm with a second engagement element arranged at a distal end of the second arm, wherein the second engagement element is rotatably and releasably coupled with the second arm and includes an actuator configured to adjust a distance between a first and second retention element such that the first and second retention elements each engage at least a portion of a second bone. The surgical system also includes a fixation device.

According to the second aspect of the present disclosure, manipulating the actuation portion increases or decreases the distance between the first and second engagement elements.

According to the second aspect of the present disclosure, the second engagement element includes a retention portion comprising a post and a threading, wherein the post is configured to releasably couple with the second arm and the threading is configured to releasably couple with the actuator.

A third aspect of the present disclosure is a surgical method. The method includes aligning a retention element having a post and a pair of extensions positioned opposite the post such that a first bone is disposed at least partially between each of the pair of extensions, releasably coupling a surgical clamp with the post of the retention element such that the post is received by a portion of a first arm of the surgical clamp, and manipulating an actuator such that each of the pair of extensions contact and releasably couple with at least a portion of the first bone. The method also includes manipulating the surgical clamp such that an engagement element disposed on a second arm of the surgical clamp contacts a surface of a second bone, coupling a first stabilization wire with the second bone, reducing an intramedullary angle between the first bone and the second bone, and derotating the second bone by manipulating the first stabilization wire. The method also includes inserting a second stabilization wire through a through hole of the second arm and into the second bone, making one or more cuts to one or more bones, and applying a fusion device across the first bone and a third bone.

A fourth aspect of the present disclosure is surgical system. The system includes a clamp. The clamp includes a first portion having a first actuator configured to interface with a first metatarsal of a patient, wherein a range of movement of the actuator is defined by an aperture, and a second portion having an extension with a retention mechanism configured to interface with a second metatarsal of the patient. The second portion is releasably coupled with the first portion and the first and second portions are translatable relative to one another via a second actuator. The system also includes stabilization wires configured to be received by at least one of the first portion and the second portion.

According to the fourth aspect of the present disclosure, the first actuator comprises a first bore disposed about a longitudinal axis of the actuator.

According to the fourth aspect of the present disclosure, the first bore is configured to receive one of the plurality of stabilization wires into and through the bore such that the stabilization wire releasably couples the first actuator with the first metatarsal.

According to the fourth aspect of the present disclosure, manipulation of the first actuator within the aperture applies a rotational force to the first metatarsal via the stabilization wire so as to manipulate the first metatarsal from a first position to a second position.

According to the fourth aspect of the present disclosure, manipulation of the first actuator within the aperture moves the stabilization wire from a first position forming a first angle with the transverse plane to a second position forming a second position forming a second angle with the transverse plane, wherein the first angle is different than the second angle.

According to the fourth aspect of the present disclosure, the first angle is greater than the second angle.

According to the fourth aspect of the present disclosure, the retention mechanism is disposed at a distal end of the extension and comprises a pair of curved protrusions defining a recess therebetween.

According to the fourth aspect of the present disclosure, the protrusions are curved in the direction of the first portion of the clamp.

According to the fourth aspect of the present disclosure, the second actuator is disposed at an end of the second portion opposite the first portion.

According to the fourth aspect of the present disclosure, the second portion includes a bore extending through the second portion along a longitudinal axis thereof.

According to the fourth aspect of the present disclosure, the second actuator includes a protrusion having a first coupling mechanism, the protrusion configured to be received by and extend through the bore, wherein the protrusion is configured to releasably couple with the second portion via the bore.

According to the fourth aspect of the present disclosure, the first portion includes a recess comprising a second coupling mechanism, the recess configured to receive and releasably couple with a portion of the protrusion of the second actuator.

According to the fourth aspect of the present disclosure, the recess of the first portion and the bore of the second portion share a common axis of the protrusion when both are releasably coupled with the protrusion of the second actuator.

According to the fourth aspect of the present disclosure, manipulation of the second actuator translates the first portion relative to the second portion about the shared axis.

According to the fourth aspect of the present disclosure, the first and second actuators each include a texture disposed on one or more outer surfaces thereof.

According to the fourth aspect of the present disclosure, the second portion includes one or more protuberances each having an aperture configured to receive one of the of stabilization wires.

A fifth aspect of the present disclosure is a surgical clamp. The surgical clamp includes a first portion having a substantially curved geometry. The first portion includes a recess positioned at a proximal end of the first portion, an elongated aperture positioned at a distal end of the first portion, and a first actuator disposed at least partially within the elongated aperture such that the elongated aperture defines a range of movement of the first actuator, wherein the first actuator includes a bore extending therethrough along a longitudinal axis thereof and configured to receive a stabilization wire. The surgical clamp also has a second portion, which includes an extension having a pair of protrusions at a distal end thereof, a bore extending along a longitudinal axis of the second portion, and a second actuator including a coupling mechanism configured to be received through and releasably couple with the bore, wherein the coupling mechanism is further configured to be received by releasably and couple with the recess such that manipulation of the second actuator translates the first portion and the second portion about the longitudinal axis of the second portion.

According to the fifth aspect of the present disclosure, the first portion includes at least one bore configured to receive a stabilization wire.

According to the fifth aspect of the present disclosure, the second portion includes at least aperture configured to receive a stabilization wire.

A sixth aspect of the present disclosure is a method of performing a Lapidus joint procedure. The method includes coupling a first portion of a device to a second metatarsal with a first and second stabilization wire, and coupling a second portion of the device to the first metatarsal with a third stabilization wire, wherein the third stabilization wire is received by a first actuator of the second portion. The method also includes manipulating a second actuator disposed on the first portion of the device so as to decrease the distance between the first and second metatarsals, and manipulating the first actuator so as rotate the first metatarsal. The method further includes coupling a surgical guide adjacent the Lapidus joint with a fourth stabilization wire, preparing a distal portion of a medial cuneiform and a proximal portion of the first metatarsal for fusion such that the distal portion of the medial cuneiform has a surface that is substantially parallel to a proximal surface of the first metatarsal, and applying a fusion device across the Lapidus joint.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front perspective view of an exemplary system for implementation in performing a surgical procedure, in accordance with the present disclosure;

FIG. 2 is a front view of the system of FIG. 1, in accordance with the present disclosure;

FIG. 3 is rear perspective of the system of FIG. 1, in accordance with the present disclosure;

FIG. 4 is a top view of the system of FIG. 1 shown adjacent to a portion of the anatomy, in accordance with the present disclosure;

FIG. 5 is a front perspective view of the system of FIG. 1 shown adjacent a portion of the anatomy, in accordance with the present disclosure;

FIG. 6 is a front perspective view of the system of FIG. 1 shown adjacent a portion of the anatomy, in accordance with the present disclosure;

FIG. 7 is a front-right perspective view of an exemplary system for implementation in performing a surgical procedure, in accordance with the present disclosure;

FIG. 8 is a top view of the system of FIG. 7, in accordance with the present disclosure;

FIG. 9 is a front view of the system of FIG. 7, in accordance with the present disclosure;

FIG. 10 is a rear perspective view of the system of FIG. 7, in accordance with the present disclosure;

FIG. 11 is a front perspective view of an exemplary retention mechanism for implementation in performing a surgical procedure, in accordance with the present disclosure;

FIG. 12 is front view of the mechanism of FIG. 11, in accordance with the present disclosure;

FIG. 13 is bottom perspective view of the mechanism of FIG. 11, in accordance with the present disclosure;

FIG. 14 is a top perspective view of the mechanism of FIG., in accordance with the present disclosure;

FIG. 15 is a front view of a portion of the mechanism of FIG. 11, in accordance with the present disclosure;

FIG. 16 is a top perspective view of an additional portion of the mechanism of FIG. 11, in accordance with the present disclosure;

FIG. 17 is a front view of an exemplary mechanism for implementation in performing a surgical procedure, in accordance with the present disclosure;

FIG. 18 is a front perspective view of the mechanism of FIG. 17, in accordance with the present disclosure;

FIG. 19 is bottom perspective view the mechanism of FIG. 17, in accordance with the present disclosure;

FIG. 20 is a top perspective view of the mechanism of FIG. 17, in accordance with the present disclosure;

FIG. 21 is a front view of a portion of the mechanism of FIG. 17, in accordance with the present disclosure;

FIG. 22 is a top perspective view of a portion of the mechanism of FIG. 17, in accordance with the present disclosure;

FIG. 23 is a front perspective view of an exemplary mechanism for implementation in performing a surgical procedure, in accordance with the present disclosure;

FIG. 24 is a bottom perspective view of the mechanism of FIG. 23, in accordance with the present disclosure;

FIG. 25 is a top view of the mechanism of FIG. 23, in accordance with the present disclosure;

FIG. 26 is a front view of a portion of the mechanism of FIG. 23, in accordance with the present disclosure;

FIG. 27 is a top view of an exemplary system for implementation in performing a surgical procedure, in accordance with the present disclosure;

FIG. 28 is a top perspective view of the system of FIG. 27, in accordance with the present disclosure;

FIG. 29 is a front view of the system of FIG. 27, in accordance with the present disclosure;

FIG. 30 is an alternate front view of the system of FIG. 27, in accordance with the present disclosure;

FIG. 31 is a side perspective view of the system of FIG. 27, in accordance with the present disclosure;

FIG. 32 is a top view of the system of FIG. 27, in accordance with the present disclosure;

FIG. 33 is a front view of a retention element of the system of FIG. 27, in accordance with the present disclosure;

FIG. 34 is a side view of the retention element of FIG. 33, in accordance with the present disclosure;

FIG. 35A is a flowchart showing steps of a method for implementation in performing a surgical procedure, in accordance with the present disclosure;

FIG. 35B is a flowchart showing subsequent steps of a method for implementation in performing a surgical procedure from FIG. 35A, in accordance with the present disclosure;

FIG. 36 is a side perspective view of a system for positioning an orthopedic implant, in accordance with the present disclosure;

FIG. 37 is an alternate side perspective view of the system of FIG. 36 for positioning an orthopedic implant, in accordance with the present disclosure;

FIG. 38 is a side cross-sectional view of the system of FIG. 36 for positioning an orthopedic implant, in accordance with the present disclosure;

FIG. 39 is a front perspective view of an exemplary system for implementation in performing a surgical procedure, in accordance with the present disclosure;

FIG. 40 is a rear perspective view of the system of FIG. 39, in accordance with the present disclosure;

FIG. 41 is right side view the system of FIG. 39, in accordance with the present disclosure;

FIG. 42 is a left side view of the system of FIG. 39, in accordance with the present disclosure;

FIG. 43 is a top view of the system of FIG. 39, in accordance with the present disclosure;

FIG. 44 is a bottom view of the system of FIG. 39, in accordance with the present disclosure;

FIG. 45 is a rear view of the system of FIG. 39, in accordance with the present disclosure;

FIG. 46 is a front view of the system of FIG. 39, in accordance with the present disclosure;

FIG. 47 is a rear perspective view of the system of FIG. 39 shown relative to anatomical structures of the foot, in accordance with the present disclosure;

FIG. 48 is an additional rear perspective view of the system of FIG. 47 shown relative to anatomical structures of the foot, in accordance with the present disclosure;

FIG. 49 is a rear perspective view of the system of FIG. 39 shown relative to anatomical structures of the foot, in accordance with the present disclosure;

FIG. 50 is a rear perspective view of the system of FIG. 39 including an exemplary surgical guide shown relative to anatomical structures of the foot, in accordance with the present disclosure;

FIG. 51 is an additional rear perspective view of the system of FIG. 39 including the surgical guide of FIG. 50 shown relative to anatomical structures of the foot, in accordance with the present disclosure;

FIG. 52 is a rear perspective view of the exemplary system of FIG. 39 including an exemplary surgical guide shown relative to anatomical structures of the foot, in accordance with the present disclosure;

FIG. 53 is an elevated rear perspective view of the system of FIG. 39 shown relative to anatomical structures of the foot, in accordance with the present disclosure;

FIG. 54 is a perspective view of a stabilization system for facilitating implementation of a fixation system, in accordance with the present disclosure;

FIG. 55 is a front perspective view of an exemplary system for implementation in performing a surgical procedure, in accordance with the present disclosure;

FIG. 56 is a rear perspective view of the system of FIG. 55, in accordance with the present disclosure;

FIG. 57 is right side view of the system of FIG. 55, in accordance with the present disclosure;

FIG. 58 is a left side view of the system of FIG. 55, in accordance with the present disclosure;

FIG. 59 is a top view of the system of FIG. 55, in accordance with the present disclosure;

FIG. 60 is a bottom view of the system of FIG. 55, in accordance with the present disclosure;

FIG. 61 is a rear view of the system of FIG. 55, in accordance with the present disclosure;

FIG. 62 is a front view of the system of FIG. 55, in accordance with the present disclosure;

FIG. 63 is an exploded view of the system of FIG. 55, in accordance with the present disclosure;

FIG. 64 is a perspective view of an exemplary embodiment of a surgical guide, in accordance with the present disclosure;

FIG. 65 is a side view of the surgical guide of FIG. 64, in accordance with the present disclosure; and

FIG. 66 is a flowchart of a method for performing a Lapidus joint procedure, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in International PCT Application No. PCT/US2018/20046, filed on Feb. 27, 2018, and entitled Intramedullary Nail Alignment Guides, Fixation Guides, Devices, Systems, and Methods of Use; International PCT Application No. PCT/US2018/64368, filed on Dec. 17, 2018, and entitled Alignment Guides, Cut Guides, Systems and Methods of Use and Assembly; International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Pat. No. D720,456 filed on Jul. 26, 2012 and entitled Lapidus Bone Wedge; and/or U.S. Pat. No. D765,844 filed on Oct. 23, 2014 and entitled Bone Plate; and/or U.S. Pat. No. D695,402 filed on Dec. 10, 2013 and entitled Lapidus Cut Guide; and/or U.S. Pat. No. D904,2016 filed on Nov. 22, 2017 and entitled Intramedullary Fastener; and/or U.S. Pat. No. D865,173 filed on Jul. 9, 2018 and entitled Cut Guide; and/or U.S. patent application Ser. No. 29/686,941 filed on Apr. 9, 2019 and entitled Cut Guide; and/or U.S. Pat. No. D904,609 filed on Apr. 9, 2019 and entitled Cut Guide; and/or United States Patent No. D9042010 filed on Apr. 9, 2019 and entitled Cut Guide; which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in United Stated Provisional Application No. 63/173,043, filed Apr. 9, 2021 and entitled Surgical Instruments, Guides, and Methods of Use; and/or International PCT Application No. PCT/US2018/20046, filed on Feb. 27, 2018, and entitled Intramedullary Nail Alignment Guides, Fixation Guides, Devices, Systems, and Methods of Use; and/or International PCT Application No. PCT/US2018/64368, filed on Dec. 17, 2018, and entitled Alignment Guides, Cut Guides, Systems and Methods of Use and Assembly; and/or International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Pat. No. D720,456 filed on Jul. 26, 2012 and entitled Lapidus Bone Wedge; and/or U.S. Pat. No. D765,844 filed on Oct. 23, 2014 and entitled Bone Plate; and/or U.S. Pat. No. D695,402 filed on Dec. 10, 2013 and entitled Lapidus Cut Guide; and/or U.S. Pat. No. D904,2016 filed on Nov. 22, 2017 and entitled Intramedullary Fastener; and/or U.S. Pat. No. D865,173 filed on Jul. 9, 2018 and entitled Cut Guide; and/or U.S. patent application Ser. No. 29/686,941 filed on Apr. 9, 2019 and entitled Cut Guide; and/or U.S. Pat. No. D904,609 filed on Apr. 9, 2019 and entitled Cut Guide; and/or United States Patent No. D9042010 filed on Apr. 9, 2019 and entitled Cut Guide; which are hereby incorporated herein by reference in their entireties.

Procedures to address deformities such as bunions and anatomical structures of and around the Lapidus joint frequently require the positioning/repositioning and/or rotation/derotation of the first metatarsal. Referred to herein as the “Lapidus” joint, this joint may also be known and referred to as the first tarsometatarsal joint. It is common for a procedure of the Lapidus joint (e.g., fusion/arthrodesis) to require that the first metatarsal be manipulated by applying one or more forces to the first metatarsal. In some procedures, this manipulation is necessary before any cutting and/or preparation and subsequent fusion of the Lapidus joint can take place. In evaluating a Lapidus joint deformity, two different criteria are typically analyzed for correction. One of these criteria is the intramedullary angle formed between the longitudinal axes of the first metatarsal and the second metatarsal. Bunion deformities and other conditions of the Lapidus joint often include the first metatarsal shifting medially from a normal anatomical position, thus increasing the IM angle between the first and second metatarsals from what can be considered an anatomically correct range of angle measures. Rotation of the first metatarsal is also analyzed, as bunion deformities and other conditions of the Lapidus joint commonly include a first metatarsal that has rotated substantially in the frontal plane in a substantially clockwise direction (when viewed from an anterior to posterior direction). Commonly, a Lapidus joint procedure such as those mentioned previously requires manipulation of the first metatarsal so as to a) correct (e.g., decrease) the IM angle between the first and second metatarsals by applying a substantially lateral force to the first metatarsal; and/or b) derotate the first metatarsal which as rotated from a normal anatomical position by applying a rotational force in a substantially counterclockwise direction when the first metatarsal is viewed in an anterior to posterior direction.

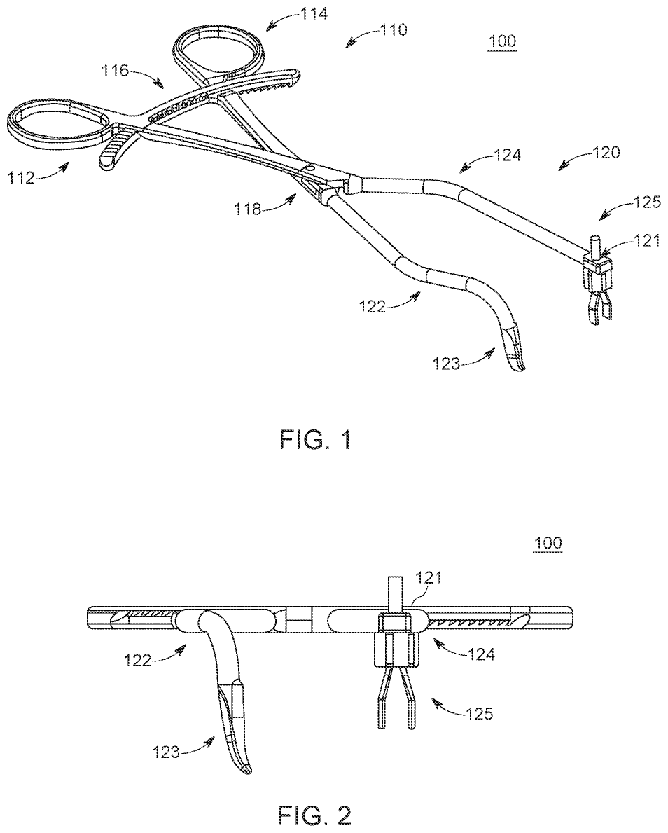

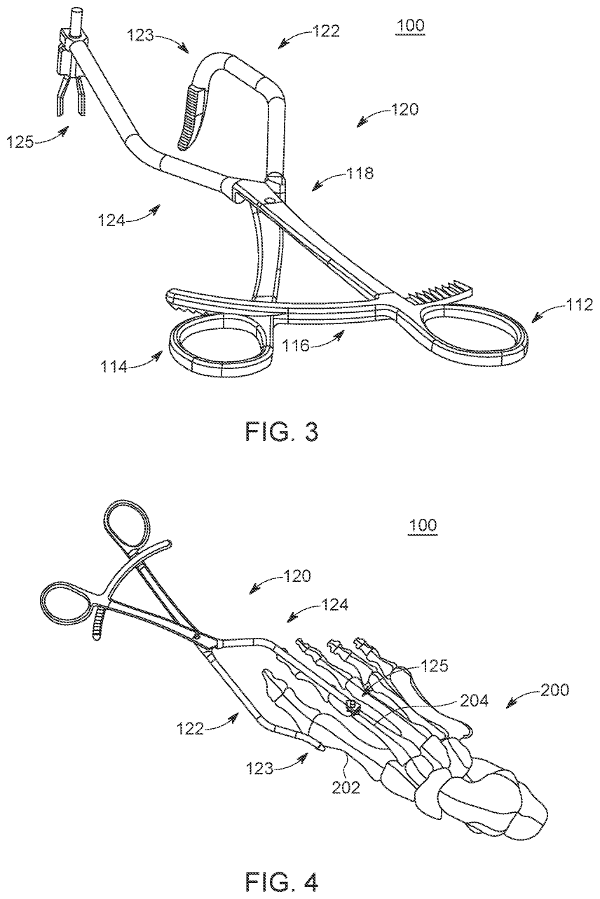

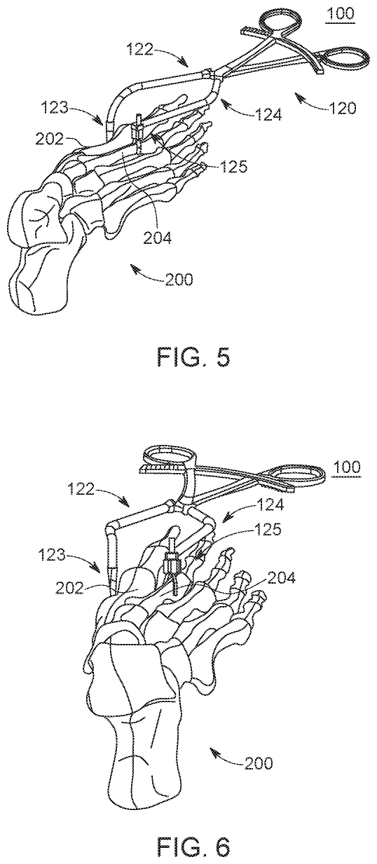

Referring to the drawings, wherein like reference numerals are used to indicate like or analogous components throughout the several views, and with particular reference to FIGS. 1-6, there is illustrated an exemplary embodiment of a clamp 100 for implementation in performing a procedure on one or more anatomical structures of the foot (for example, procedures of the Lapidus joint such as arthrodesis or other similar fusion procedures). In some aspects, the clamp 100 may be manipulated such that one or more components of the clamp contact one or more anatomical structures of the foot and, when force is applied to the clamp by a physician, at least one of the anatomical structures is selectively repositioned by the physician. The clamp 100, as shown and described herein with reference to FIGS. 1-6, includes an upper portion 110 (e.g., handle, etc.) arranged substantially opposite the clamp 100 from a lower portion 120 (e.g., engagement portion, coupling portion, etc.). Positioned between the upper portion 110 and the lower portion 120 is a coupling 118. The clamp 100 as shown includes a pair of members pivotably coupled by the coupling 118, where each member of the pair of members includes at least a portion of the upper portion 110 and a portion of the lower portion 120. A rachet 116 may be configured to adjust the clamp from a first position to a second position, or to retain the clamp in a desired position (e.g., to maintain some desired correction). In some aspects, the coupling 118 may include a geometry that defines a range of motion of the upper portion 110 and/or the lower portion 120 of the clamp 100, for example a ratchet as shown or other coupling mechanism.

The clamp 100 is further shown to include, on the lower portion of the clamp 120, a first arm 122 and a second arm 124 extending from the coupling 118. As shown, the first and second arm 122, 124 are integral with first and second handle portions 112 and 114 (where the first handle portion 112 is integral with the second arm 124, and the second handle portion 114 is integral with the first arm 122). The first arm 122 is shown to include a bend disposed between the coupling 118 and an interface component 123, where the interface component 123 is disposed at a terminal end of the first arm 122. As shown, the interface component 123 is positioned substantially perpendicular to the first arm 122 (e.g., bent in the plantar direction) between the coupling 118 and the aforementioned bent (but may be arranged alternatively in some aspects). The interface component 123 is further shown to have a curvature configured to accommodate the curvature of the medial portion of a first metatarsal 202 as shown and described with reference to at least FIGS. 4-6. In some aspects, the interface component 123 may be configured to interface with the distal portion of the first metatarsal 202. The interface portion 123 may also include a textured surface on at least one surface thereof (e.g., the surface with the concave curvature to accommodate the first metatarsal 202) so as to promote prolonged contact with the surface of the first metatarsal 202 by increasing friction between the surfaces.

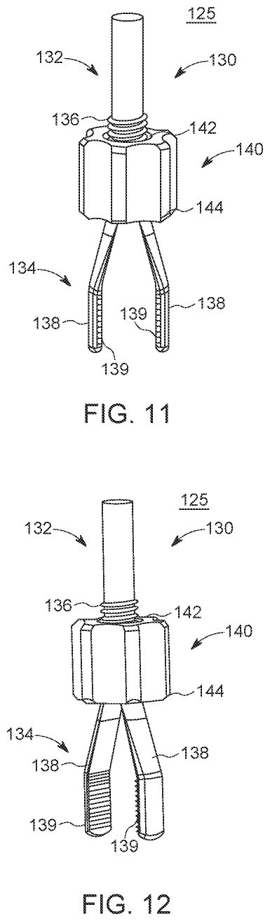

The second arm 124 is shown to include a retention element 125 arranged at a terminal end of the second arm 124, where the retention element is releasably couplable with the second arm 124. The retention element is shown to include a post 132 (seen in at least FIGS. 11-12) that is configured to be received (moving in a plantar to dorsal direction) by an opening in the second arm 124. The retention element 123 is configured to facilitate coupling with a second metatarsal 204, in some aspects at a distal portion thereof. The retention element 123 is configured to be pivotable within the opening of the second arm 124 so as to promote coupling with the second metatarsal 204 despite various lateral geometries of the second metatarsal 204.

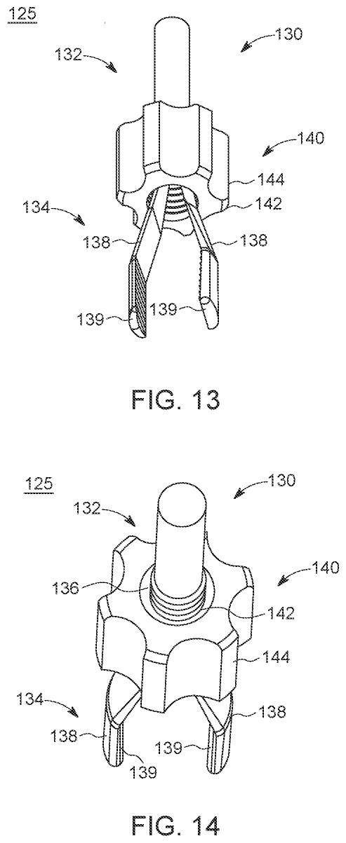

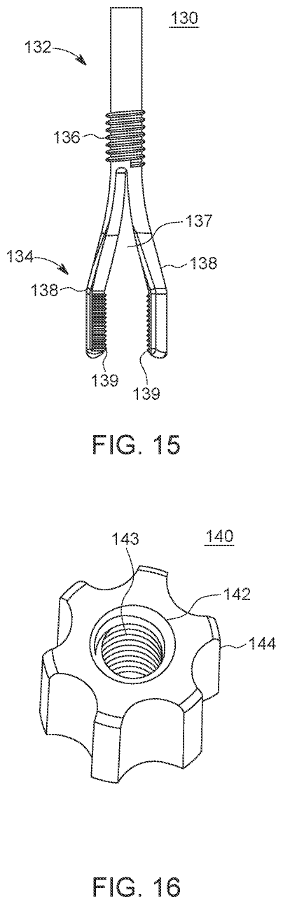

The retention element 125, which is shown further in FIGS. 11-16, includes a first element 130 and a second element 140. The first element 130 includes a post 132 (e.g., upper portion) on an upper portion thereof and a pair of engagement elements 134 disposed on a lower portion thereof (e.g., opposite the post 132). The post 132 as shown includes a threading 136 disposed on at least a portion thereof. As shown, the threading 136 is disposed on a lower portion of the post 132. The engagement elements 134 as shown are a pair of prongs extending from the lower portion of the post 132 adjacent the lower portion of the threading 136. The engagement elements 134 include a first segment 138 which extends from the lower portion of the post 132 at an orthogonal angle. The engagement elements 134 further include a second segment 139 which extends from the first segment 138 and continues to the terminal end of the engagement elements 134. As shown, the second segments 139 are substantially parallel to one another. The interior surfaces of the second segments 139 (e.g., those that face one another) include a texture configured to induce friction upon contact with a bone and promote retention of the bone therebetween.

The retention element 125 is further shown to include an actuator 140, where the actuator 140 is configured to adjust a distance between the engagement elements 134 so as to position the engagement elements 134 adjacent the second metatarsal 204 and, ultimately, be adjusted further to initiate contact (e.g., engagement, releasable coupling, etc.) with the second metatarsal 204. The actuator 140 includes an opening 142 configured to receive the post 132 into and through the opening 142. The opening 142 further includes a threading 143 disposed on an interior surface of the opening 142 with a geometry complimentary to that of the threading 136 so as to facilitate threadable, releasable coupling. The actuator 140 also includes a texture 144 disposed on an exterior surface thereof to facilitate gripping and manipulation of the actuator 140. The actuator 140 is configured that once the first element 130 has been positioned such that the engagement elements 134 are arranged adjacent the second metatarsal 204, the actuator may be releasably coupled with the post 132 such that the threading 136 and 143 engage and translate the actuator 140 down the threading 136 of the post 132. As the actuator 140 begins to contact the engagement elements 134, the engagement elements 134 may be driven closer together by a force applied by the actuator 140 (e.g., the actuator 140 begins to receive at least a portion of the engagement elements 134 within the opening 142 of the actuator 140 thus forcing the engagement elements 134 closer together). Accordingly, the space between the engagement elements 134 decreases and the medial and lateral surfaces of the second metatarsal 204 are gripped by the second segments 139 of the retention element 125. The clamp 100 may then be releasably coupled with the post 132 of the retention element 125 by positioning the clamp such that the opening of the second arm 124 received at least a portion of the post 132.

The clamp 100 is configured such that when the interface portion 123 abuts the first metatarsal 202 from the medial side and the retention element 125 engages the second metatarsal 204 from both medial and lateral sides, a force may be applied to at least the medial side of the first metatarsal 202 by manipulating the upper portion 110 such that the distance between the terminal ends of the first and second arms 122, 124 is decreased. Accordingly, such a force decreases an intramedullary angle of the first and second metatarsals 202, 204 by positioning at least the distal portion of the first metatarsal 202 closer to the second metatarsal 204 than prior to the procedure. Further, the clamp 100 is configured such that, when engaged as described previously, a space remains between the first and second arms 122, 124 so as to allow for manipulation of the hallux (e.g., “big toe”). Such manipulation may include repositioning of the hallux to evaluate soft tissue or otherwise manipulate anatomical structures adjacent to those of the Lapidus joint.

As shown in FIGS. 4-6, the clamp 100 is shown engaged with a schematic diagram of a foot 200. The interface element 123 is shown to be engaged with a distal portion of the first metatarsal 202 while the retention element 125 and portions thereof are engaged with a distal portion of the second metatarsal 204. As shown, the upper portion 110 of the clamp 100 may be manipulated such that a force is applied to the medial surface of the first metatarsal 202 so as to reduce an intramedullary angle formed between central longitudinal axes of the first and second metatarsals 202, 204. Once the desired correction to the intramedullary angle has been achieved, the rachet 116 may be engaged so as to retain the clamp in the desired position such that the clamp 100 retains the position that is providing the desired correction.

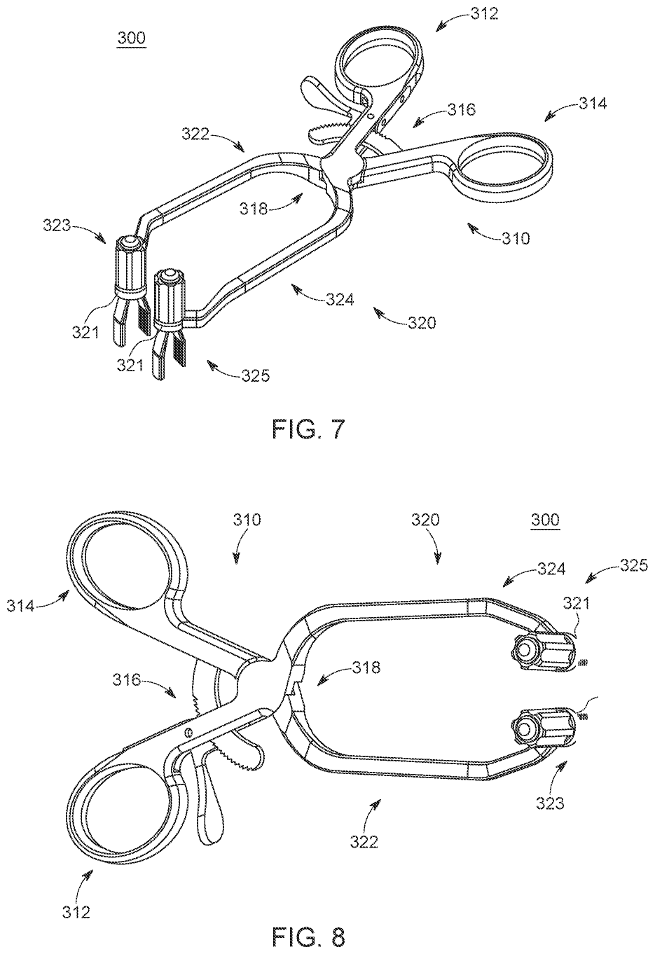

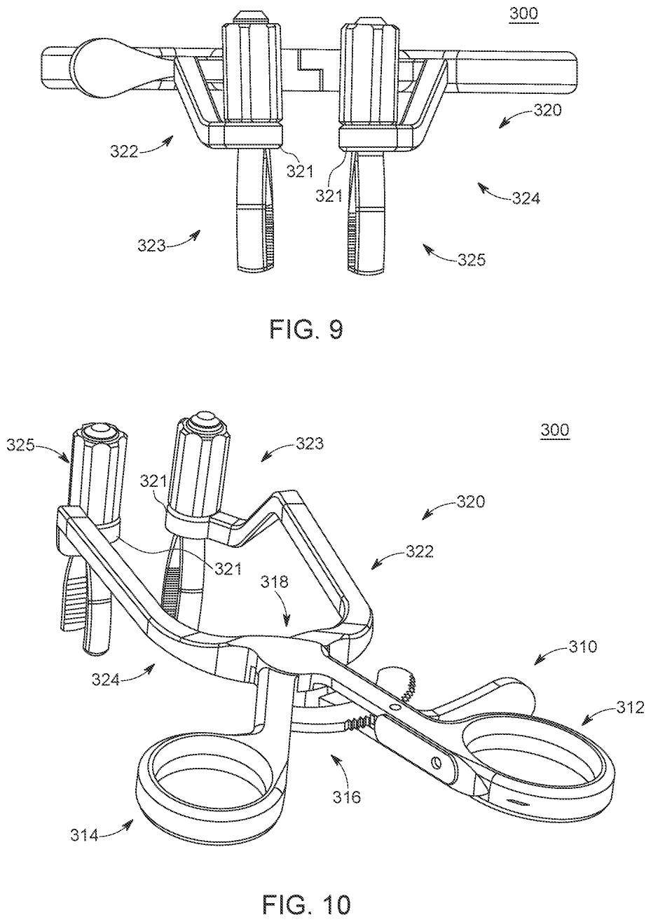

Referring now to FIGS. 7-10, an alternate clamp 300 is shown. The clamp 300 may be implemented for the same and/or similar procedures as the clamp 300 or as other clamps shown and described subsequently herein. The clamp 300 includes an upper portion 310 (e.g., handle, etc.) arranged substantially opposite the clamp 300 from a lower portion 320 (e.g., engagement portion, coupling portion, etc.). Positioned between the upper portion 310 and the lower portion 320 is a coupling 318. The clamp 300 as shown includes a pair of members pivotably coupled by the coupling 318, where each member of the pair of members includes at least a portion of the upper portion 310 and a portion of the lower portion 320. A rachet 316 may be configured to adjust the clamp from a first position to a second position, or to retain the clamp in a desired position (e.g., to maintain some desired correction). In some aspects, the coupling 318 may include a geometry that defines a range of motion of the upper portion 310 and/or the lower portion 320 of the clamp 300, for example a ratchet as shown or other coupling mechanism.

The clamp 300 is shown to include a first arm 322 and a second arm 324, where the first and second arms 322, 324 are substantially symmetrical. The first and second arms 322, 324 each include an opening 321 at a distal end thereof, with the openings 321 arranged slightly plantar (e.g., lower than) relative to the majority of the first and second arms 322, 324. Each of the openings may be the same as and/or similar to the opening as shown and described with reference to the clamp 100. The openings 321 of the first and second arms 322, 324 are configured to accommodate and pivotably, releasably couple with first and second retention elements 323, 325 (which as shown, are substantially the same component). The first and second retention elements are configured to interface, engage, and releasably couple with the first and second metatarsals 202, 204 respectively, as shown and described in previous figures.

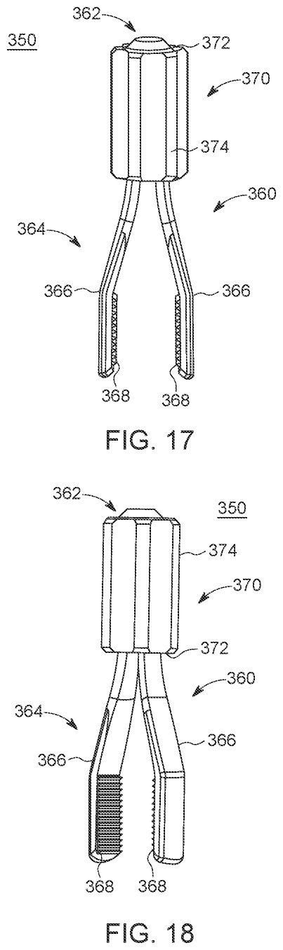

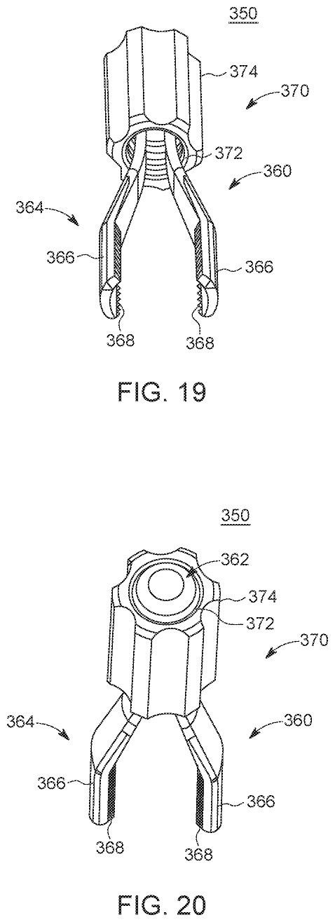

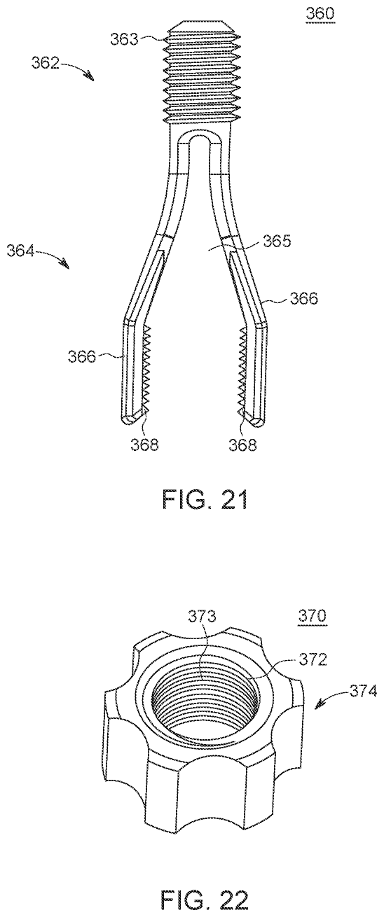

As shown further in FIGS. 17-22, the retention elements 323, 325 are each shown as a retention system 350, where each of the retention elements 323, 325 of the clamp 300 may be considered a retention system the same as shown as component 350. The retention system 350 includes a first element 360 and an actuator 370. The first element 360 includes a post 362 (e.g., upper portion) on an upper portion thereof and a pair of engagement elements 366 disposed on a lower portion 364 thereof (e.g., opposite the post 132). The post 362 as shown includes a threading 363 disposed on at least a portion thereof. As shown, the threading 363 is disposed on a lower portion of the post 362. The engagement elements 366 as shown are a pair of prongs extending from the lower portion of the post 362 adjacent the lower portion of the threading 363. The engagement elements 366 include a first segment which extends from the lower portion of the post 362 at an orthogonal angle. The engagement elements 366 further include a second segment 368 which extends from the first segment and continues to the terminal end of the engagement elements 366. As shown, the second segments 368 are substantially parallel to one another. The interior surfaces of the second segments 368 (e.g., those that face one another) include a texture configured to induce friction upon contact with a bone and promote retention of the bone therebetween.

The retention system 350 is further shown to include the actuator 370, where the actuator 370 is configured to adjust a distance between the engagement elements 366 so as to position the engagement elements 366 adjacent the second metatarsal 204 and, ultimately, be adjusted further to initiate contact (e.g., engagement, releasable coupling, etc.) with the second metatarsal 204. The actuator 370 includes an opening 372 configured to receive the post 362 into and through the opening 372. The opening 372 further includes a threading 373 disposed on an interior surface of the opening 372 with a geometry complimentary to that of the threading 363 so as to facilitate threadable, releasable coupling. The actuator 370 also includes a texture 374 disposed on an exterior surface thereof to facilitate gripping and manipulation of the actuator 370. The actuator 370 is configured that once the first element 360 has been positioned such that the engagement elements 366 are arranged adjacent the second metatarsal 204, the actuator may be releasably coupled with the post 362 such that the threading 363 and 373 engage and translate the actuator 370 down the threading 363 of the post 362. As the actuator 370 begins to contact the engagement elements 366, the engagement elements 366 may be driven closer together by a force applied by the actuator 370 (e.g., the actuator 370 begins to receive at least a portion of the engagement elements 370 within the opening 372 of the actuator 370 thus forcing the engagement elements 366 closer together and decreasing the volume of a space therebetween 365). Accordingly, the space 365 between the engagement elements 366 decreases and the medial and lateral surfaces of the second metatarsal 204 are gripped by the second segments 368 of the retention system 350. The clamp 300 may then be releasably coupled with the posts 362 of the retention systems 350 by positioning the clamp such that the openings of the first and second arm 322, 324 receive at least a portion of the posts 362.

The clamp 300 is configured such that when the first retention element 323 of the first arm 322 couples with the first metatarsal 202 from both medial and lateral sides and the second retention element 325 of the second arm 324 engages the second metatarsal 204 from both medial and lateral sides, a force may be applied to at least the medial side of the first metatarsal 202 by manipulating the upper portion 110 such that the distance between the terminal ends of the first and second arms 322, 324 is decreased. Accordingly, such a force decreases an intramedullary angle of the first and second metatarsals 202, 204 by positioning at least the distal portion of the first metatarsal 202 closer to (e.g., more lateral) the second metatarsal 204 than prior to the procedure. Further, the clamp 300 is configured such that, when engaged as described previously, a space remains between the first and second arms 322, 324 so as to allow for manipulation of the hallux (e.g., “big toe”). Such manipulation may include repositioning of the hallux to evaluate soft tissue or otherwise manipulate anatomical structures adjacent to those of the Lapidus joint.

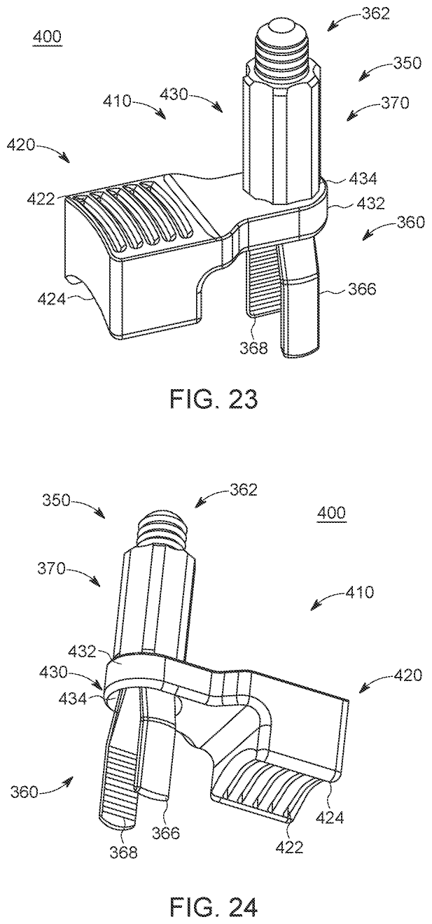

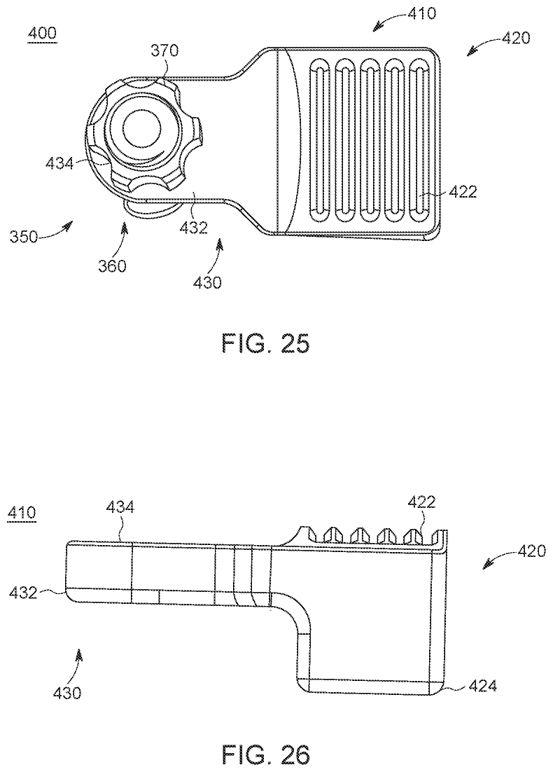

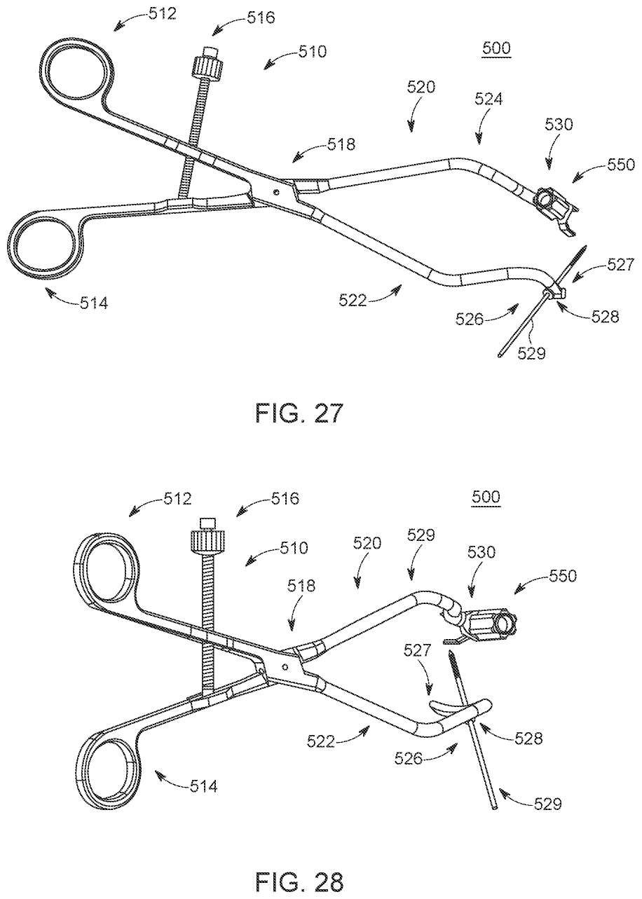

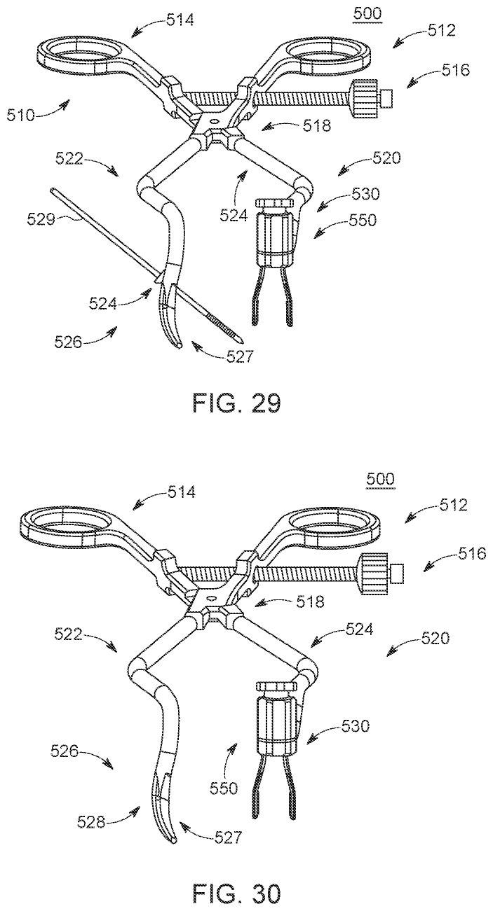

Referring now to FIGS. 23-26, a cut guide system 400 is shown which includes the retention system 350 as well as a cut guide 410. It should be noted that the cut guide 410 may also be implemented with the retention element 125 and the clamp 100. The cut guide 410 may include embodiments other than those shown herein (e.g., left/right counterparts, etc.). As shown, the cut guide 410 includes a cutting portion 420 and a coupling portion 430, where the coupling portion 430 is a rounded protrusion from the cutting portion 420. The cut guide 410 is shown to releasably couple with the retention system 350 via the coupling portion 430 such that an opening 434 (extending from a top surface 432 thereof through the coupling portion 430 to a bottom surface) of the cut guide 410 receives the post 362 of the first element 360, with the actuator 370 then also received the post 362 so as to dispose the cut guide 410 between the engagement elements 366 and the actuator 370.

The cutting portion 420 is shown to include a plurality of cut slots 422 extending through the cutting portion 420. The cut slots 422 as shown are substantially the same size and run substantially perpendicular to one another, but may have alternate geometries and arrangement in some aspects. In some aspects, the cut guide system 400 may be configured to position the cut slots 422 substantially parallel to another surface (e.g., parallel to a surface of the proximal first metatarsal 202 that has already been cut such that a parallel cut may be made to the distal first cuneiform 204 and thus create ideal surfaces for fusion). The physician may determine which of the cut slots 422 are best positioned to make the desired cut and, once a determination is made, the physician may implement a reciprocating saw or other similar instrument to make the desired cuts.

Referring now to FIGS. 17-24, a clamp 500 is shown. The clamp 500 may be similar to the clamps 100, 300 as shown and described previously and may also be implemented for the same and/or similar procedures (e.g., those of the Lapidus joint).). In some aspects, the clamp 500 may be manipulated such that one or more components of the clamp 500 contact one or more anatomical structures of the foot and, when force is applied to the clamp by a physician, at least one of the anatomical structures is selectively repositioned by the physician. The clamp 500, as shown and described herein with reference to FIGS. 17-24, includes an upper portion 510 (e.g., handle, etc.) arranged substantially opposite the clamp 500 from a lower portion 520 (e.g., engagement portion, coupling portion, etc.). Positioned between the upper portion 510 and the lower portion 520 is a coupling 518. The clamp 500 as shown includes a pair of members pivotably coupled by the coupling 518, where each member of the pair of members includes at least a portion of the upper portion 510 and a portion of the lower portion 520. A locking mechanism 516 may be configured to adjust the clamp 500 from a first position to a second position, or to retain the clamp 500 in a desired position (e.g., to maintain some desired correction). In some aspects, the coupling 518 may include a geometry that defines a range of motion of the upper portion 510 and/or the lower portion 520 of the clamp 500, for example a ratchet as shown or other coupling mechanism.

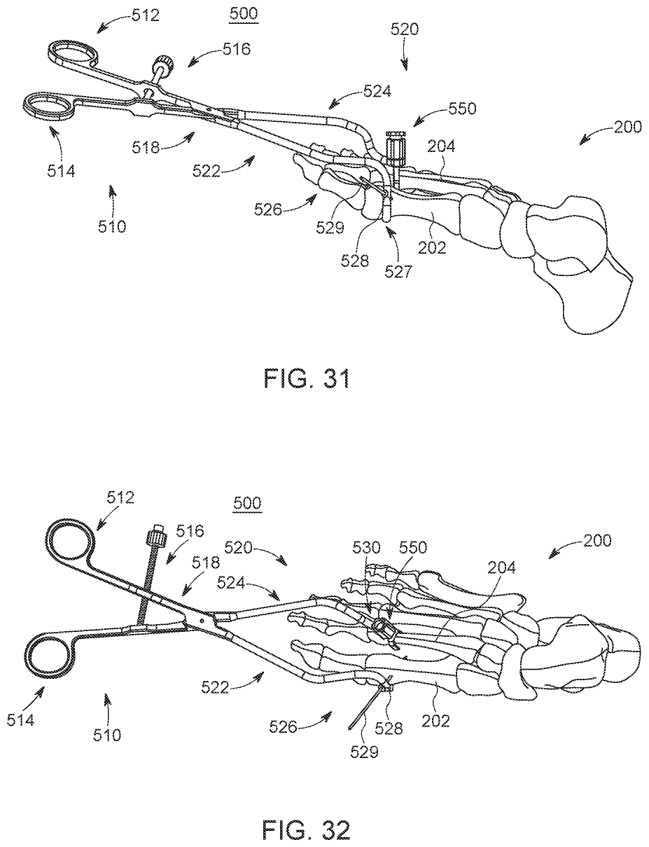

The clamp 500 is further shown to include, on the lower portion of the clamp 520, a first arm 522 and a second arm 524 extending from the coupling 518. As shown, the first and second arm 522, 524 are integral with first and second handle portions 512 and 514 (where the first handle portion 512 is integral with the second arm 524, and the second handle portion 514 is integral with the first arm 522). The first arm 522 is shown to include a bend disposed between the coupling 518 and an interface component 526, where the interface component 526 is disposed at a terminal end of the first arm 522. As shown, the interface component 526 is positioned substantially perpendicular to the first arm 522 (e.g., bent in the plantar direction) between the coupling 518 and the aforementioned bend (but may be arranged alternatively in some aspects). The interface component 526 is further shown to have a curvature configured to accommodate the curvature of the medial portion of a first metatarsal 202 as shown and described with reference to at least FIGS. 31-32. In some aspects, the interface component 526 may be configured to interface with the distal portion of the first metatarsal 202. The interface portion 526 may also include a textured surface 527 on at least one surface thereof (e.g., the surface with the concave curvature to accommodate the first metatarsal 202) so as to promote prolonged contact with the surface of the first metatarsal 202 by increasing friction between the surfaces. The interface portion 526 is also shown to include an aperture 528 extending therethrough, where the aperture 528 is configured to receive a stabilization wire 529. The stabilization wire 529 may be a k-wire or, as shown, may be an olive wire with a depth stop (e.g., a portion of the wire with a diameter greater than that of the opening 528). The stabilization wire 529 may be inserted through the opening 528 and coupled with the first metatarsal 202 as shown, with the stabilization wire extending from the first metatarsal 202 at an oblique angle relative to the horizontal.

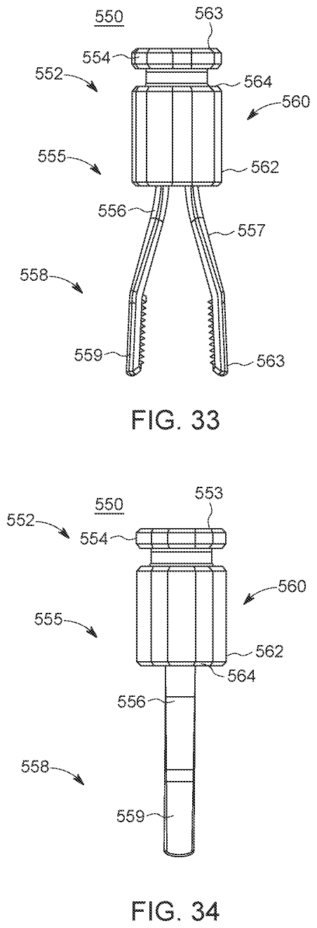

The second arm 524 is shown to include a retention element 550 arranged at a terminal end of the second arm 524, where the retention element 550 is releasably couplable with the second arm 524. The retention element 550 is shown to include a post 552 (seen in at least FIGS. 33-34) that is configured to be received (moving in a plantar to dorsal direction) by an opening in the second arm 524. The retention element 550 is configured to facilitate coupling with a second metatarsal 204, in some aspects at a distal portion thereof. The retention element 550 is configured to be pivotable within the opening 530 of the second arm 524 so as to promote coupling with the second metatarsal 204 despite various lateral geometries of the second metatarsal 204.

The retention 550, which is shown further in FIGS. 33-34, includes a first element 552 and a second element 560. The first element 552 includes a post 555 (e.g., upper portion) on an upper portion thereof and a pair of engagement elements 559, 563 disposed on a lower portion thereof (e.g., opposite the post 555). The post 555 includes a threading (not shown) disposed on at least a portion thereof, which may be the same as and/or similar to that shown with other similar embodiments disclosed previously herein. The threading is disposed on a lower portion of the post 555. The engagement elements 559, 563 as shown are a pair of prongs extending from the lower portion of the post 555 adjacent the lower portion of the threading. The engagement elements 559, 563 include a pair of extensions 556, 557 which extends from the lower portion of the post 555 (adjacent the threading) at an orthogonal angle. The engagement elements 559, 563 include a bend (also at an orthogonal angle) along a portion thereof, then continue substantially parallel to one another between the bend and the terminal end of each of the engagement elements 559, 563. The interior surfaces of the engagement elements 559, 563 (e.g., those that face one another) include a texture configured to induce friction upon contact with a bone and promote retention of the bone therebetween.

The retention element 550 is further shown to include an actuator 560, where the actuator 560 is configured to adjust a distance between the engagement elements 559, 563 so as to position the engagement elements 559, 563 adjacent the second metatarsal 204 and, ultimately, be adjusted further to initiate contact (e.g., engagement, releasable coupling, etc.) with the second metatarsal 204. The actuator 560 includes an opening 553 configured to receive the post 555 into and through the opening 553. The opening 553 further includes a threading disposed on an interior surface of the opening 553 with a geometry complimentary to that of the threading of the post 555 so as to facilitate threading and releasable coupling therebetween. The actuator 560 also includes a first texture 554 disposed on an upper portion of exterior surface thereof to facilitate gripping and manipulation of the actuator 560. The actuator 560 also includes a second texture 562 (larger than the first texture 554) which is disposed below a depression extending the circumference (e.g., or another outer dimension) of the actuator 560 and disposed below the first texture 554 and above the second texture 564. The actuator 560 is configured that once the first element 552 has been positioned such that the engagement elements 559, 563 are arranged adjacent the second metatarsal 204, the actuator may be releasably coupled with the post 555 such that the threading of the post and the opening 553 of the actuator 560 engage and translate the actuator 560 down the threading of the post 555. As the actuator 560 begins to contact the extensions 556, 557, the extensions 556, 557 (and accordingly the engagement elements 559, 563) may be driven closer together by a force applied by the actuator 560 (e.g., the actuator 560 begins to receive at least a portion of the extensions 556, 557 within the opening 553 of the actuator 560 thus forcing the engagement elements 559, 563 closer together). Accordingly, the space between the engagement elements 559, 563 decreases and the medial and lateral surfaces of the second metatarsal 204 are gripped by the 559, 563 of the retention element 550, The clamp 500 may then be releasably coupled with the post 555 of the retention element 550 by positioning the clamp such that the opening of the second arm 524 received at least a portion of the post 555.

The clamp 500 is configured such that when the interface portion 526 abuts the first metatarsal 202 from the medial side and the retention element 550 engages the second metatarsal 204 from both medial and lateral sides, a force may be applied to at least the medial side of the first metatarsal 202 by manipulating the upper portion 510 such that the distance between the terminal ends of the first and second arms 522, 524 is decreased. Accordingly, such a force decreases an intramedullary angle of the first and second metatarsals 202, 204 by positioning at least the distal portion of the first metatarsal 202 closer to the second metatarsal 204 than prior to the procedure. Further, the clamp 500 is configured such that, when engaged as described previously, a space remains between the first and second arms 522, 524 so as to allow for manipulation of the hallux (e.g., “big toe”). Such manipulation may include repositioning of the hallux to evaluate soft tissue or otherwise manipulate anatomical structures adjacent to those of the Lapidus joint.

As shown in FIGS. 31-32, the clamp 500 is shown engaged with a schematic diagram of a foot 200. The interface element 526 is shown to be engaged with a distal portion of the first metatarsal 202 while the retention element 550 and portions thereof are engaged with a distal portion of the second metatarsal 204. As shown, the upper portion 510 of the clamp 500 may be manipulated such that a force is applied to the medial surface of the first metatarsal 202 so as to reduce an intramedullary angle formed between central longitudinal axes of the first and second metatarsals 202, 204. Once the desired correction to the intramedullary angle has been achieved, the rachet 516 may be engaged so as to retain the clamp in the desired position such that the clamp 500 retains the position that is providing the desired correction.

It should be noted that the clamp 500 and components thereof may be the same as and/or similar to those shown and described previously with reference to the clamps 100 and 300, as well as components that may be implemented in conjunction with the clamps 100 and 300. For example, the retention element 550 may be replaced by the retention element 125, or vice-versa. Similarly, the retention element 550 and components thereof may be configured to releasably couple with the guide system 400 of FIGS. 23-26, or other similar guide or outrigger systems.

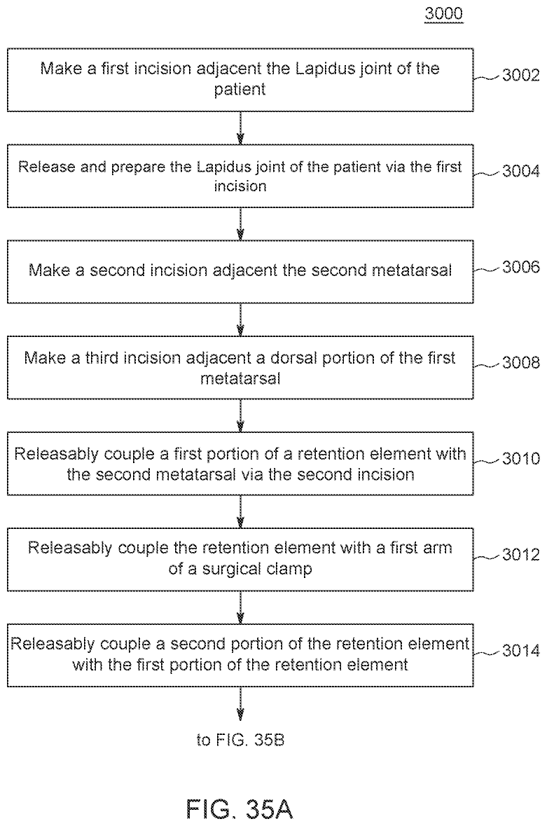

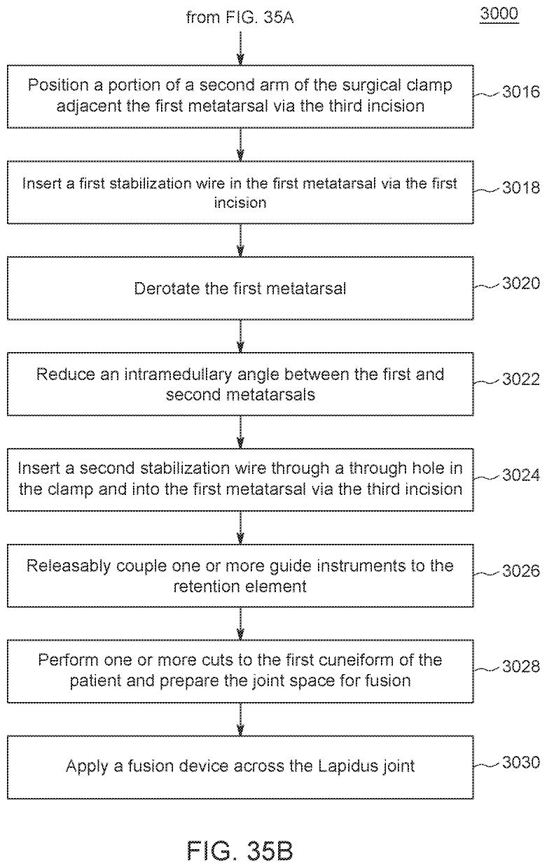

Referring now to FIGS. 35A-B, a process or method 3000 is shown for performing a surgical procedure, for example a fusion of the Lapidus joint (e.g., first tarsometatarsal joint, a bunion correction procedure, etc.). The process 3000 and the steps thereof may be performed in one or more alternative sequences to that shown in FIGS. 35A-B. The steps of the process 3000 may also be omitted, repeated, or performed in an alternate sequence to that shown. In some aspects, the process 3000 may also include performing one or more additional steps not shown in FIGS. 35A-B.

The process 3000 is shown to include a step 3002 making a first incision adjacent the Lapidus joint of a patient, according to some aspects. In some aspects, the incision of the step 3002 may be made at a position substantially dorsal-medial to the Lapidus joint. Further, the incision of the step 3002 may be made using one or more instruments (e.g., scalpel or other cutting instrument) provided as part of a surgical kit for performing a procedure such as that shown and described with reference to the process 3000 and FIGS.

The process 3000 is shown to include a step 3004 releasing and preparing the Lapidus joint of the patient via the first incision, according to some aspects. The step 3004 may include various steps and techniques commonly implemented for releasing preparing a joint for an arthrodesis procedure such as that of the process 3000. For example, the step 3004 may include releasing the Lapidus joint such that the first metatarsal is more easily manipulated in subsequent steps of the process 3000. In some aspects, a cut may be made at the base (e.g., proximal end) of the first metatarsal using a cut guide the same as or similar to those shown and described herein previously. It should also be understood that, in some aspects, a cut may also be made to the cuneiform in the step 3004, with the cut to the cuneiform made before or after any cut to the first metatarsal. In some aspects, the step 3004 may include a cut to the cuneiform with any cut to the first metatarsal made in a previous or subsequent step. After performing such a cut, a fragment of the first metatarsal (produced as a result of the cut) is removed from the joint space. The Lapidus joint and surrounding structures may also be evaluated after release of the joint as well as the aforementioned cut, with adjustments or further manipulation common to achieve a desired result. In some aspects, the distal portion of the first metatarsal may be prepared further, for example by fenestration or other common techniques to prepare at least a portion of a joint space for fusion.

The process 3000 is shown to include a step 3006 of making a second incision adjacent the second metatarsal, according to some aspects. The incision of the step 3006 may be the same as or similar to that of the step 3008 (e.g., size, etc.). In some aspects, the second incision may be made from a substantially lateral position (e.g., direction, etc.) relative to the second metatarsal so as to facilitate access to the second metatarsal in subsequent steps of the process 3000. In some aspects, the incision of the step 3006 may be made by referencing one or more anatomical structures, for example the second metatarsal, with the incision made adjacent a distal portion thereof. As shown and described herein, the second incision may be a “stab” incision, although other incision types and techniques may be implemented by a physician.

The process 3000 is shown to include a step 3008 making a third incision adjacent a dorsal portion of the first metatarsal, according to some aspects. In some aspects, the incision of the step 3008 may be made at a distal portion of the first metatarsal (e.g., adjacent the first metatarsal head) and thus substantially opposite the first metatarsal from the first incision (in distal-proximal) direction. In some aspects, the third incision may be made from a substantially medial direction relative to the head of the first metatarsal so as to facilitate access to and manipulation of the first metatarsal in subsequent steps of the process 3000. The third incision of the step 3008 may also be made adjacent (and to provide access to at least) a dorsal surface, a lateral portion, and proximal of the head of the second metatarsal (where the head is a distal portion of the second metatarsal). The incisions of the steps 3006 and 3008 may at least partially be in a single plane in the frontal plane (e.g., are substantially equidistant from their respective proximal-most portions of the first and second metatarsals, respectively).

The process 3000 is shown to include a step 3010 releasably coupling a first portion of a retention element with the second metatarsal via the second incision, according to some aspects. In releasably coupling the first portion of the retention element, the physician takes a dorsal approach to the second metatarsal (e.g., from above) with the first portion of the retention element entering the second incision. The first portion of the retention element is then inserted until the engagement elements 559, 563 (e.g., prongs, protrusions, extensions, etc.) contact (or are positioned directly adjacent) a medial surface and a lateral surface (e.g., the flat of the second metatarsal on the lateral portion thereof). The first portion of the retention element may be, for example, the engagement elements 556 of the retention element 550 as shown in at least FIGS. 33-34, or may also include other similar portions of other retention elements as shown and described previously herein. In some aspects, the step 3010 may include identification and/or manipulation of the extensor tendon by the physician in order to place the first portion of the retention element.

The process 3000 is shown to include a step 3012 releasably coupling the retention element with a first arm of a surgical clamp, according to some aspects. The step 3012 may include the clamp 500 as shown and described previously herein as well as other clamps including but not limited to those described herein. The step 3012 may include aligning a first arm of the surgical clamp, for example the first arm of the surgical clamp (which may correspond to the second arm 524 of the clamp 500, where nomenclature of first and second arms of a clamp may be arbitrary), with the physician manipulating the first arm of the clamp such that an opening (e.g., the opening 530 of the clamp 500) with a geometry complimentary to that of an upper portion of the retention element (e.g., the post 555 of the retention element 500), where the opening includes a geometry complimentary to that of the upper portion of the retention element (e.g., the post 555). The upper portion of the retention element, when releasably coupled with the clamp, may extend into and at least partially through the opening of the clamp from an inferior to superior direction (e.g., when releasably coupling the clamp and the retention element, the clamp and the opening positioned above the upper-most portion of the retention element.

The process 3000 is shown to include a step 3014 releasably coupling a second portion (also referred to as a cap portion) of the retention element with the first portion of the retention element, according to some aspects. In some aspects, the step 3014 may include releasably coupling a cap portion of the retention element with the upper portion of the retention element so as to retain the distal portion of the first arm of the clamp substantially between the engagement elements and cap portion (and the upper-most portion of the upper portion of the retention element). In order to facilitate the coupling of the cap portion with the upper portion of the retention element (e.g., the upper portion of the post), the cap portion may include an interior threading complimentary to an exterior threading disposed on an upper portion of the post. The cap portion may be positioned superior relative to the top of the post, and then coupled via the aforementioned threading such that at least a portion of the post is disposed within the cap portion when the two components are coupled. In a coupled position, the cap portion may contact or otherwise be positioned adjacent an upper surface of the distal portion of the first arm.

Referring now to FIG. 35B, the process 3000 is shown to include a step 3016 positioning a portion of a second arm of the surgical clamp adjacent the first metatarsal via the third incision, according to some aspects. In some aspects, a third incision as described in the process 3000 may not be made, in which case the step 3016 may include positioning a portion of a second arm of the surgical clamp adjacent the first metatarsal but external the skin. The second arm of the clamp (for example, the clamp 500 as shown and described previously herein) may correspond to the first arm 522 of the clamp 500, where the nomenclature of the first and second arms is arbitrary. With regard to the step 3016, the second arm of the clamp may include one or more interfacing portions on a distal portion thereof, where the interface portion extends from a central portion of the second arm at an orthogonal or perpendicular angle in a substantially dorsal (e.g., inferior) direction. In the step 3016, this interface portion may be positioned adjacent or in contact with a medial portion of the first metatarsal by insertion through the third incision (made in the step 308). The interface portion may have a curvature along at least a portion thereof, where step 3016 may include positioning the curvature relative to the curvature of the medial side of the first metatarsal of the patient. The step 3016 may further include positioning the interface portion such that an opening therein (e.g., the same as or similar to the opening 528 of the clamp 500) is visible by the physician through the third incision or adjacent the third incision.

The process 3000 is shown to include a step 3018 inserting a first stabilization wire (e.g., k-wire, olive wire, rotation wire, lever wire, stabilization member, rotation member, etc.) in the first metatarsal via the first incision, according to some aspects. The first stabilization wire may be a traditional k-wire, an olive wire, or other stabilization wires commonly known and used in similar procedures. When inserted in the first metatarsal (at a proximal portion thereof), the first stabilization wire may protrude from the first metatarsal in a dorsal-medial direction. In some aspects, the physician may determine placement of the first stabilization wire in the dorsal-medial directions based on an estimate of desired derotation of the first metatarsal. For example, if a large degree of derotation is desired the first stabilization wire may be placed more medially than dorsally, while ifs small degree of derotation is desired the first stabilization wire may be placed more dorsally than medially.

The process 3000 is shown to include a step 3020 derotating the first metatarsal, according to some aspects. As mentioned above, step 3020 may be performed simultaneously with the step 3022 described subsequently. Further, in some aspects, the steps 3020 and 3022 may be performed in alternate orders when not performed simultaneously. In performing the step 3020, the physician manipulates the first stabilization wire placed in the step 3018 such that the first metatarsal is rotated about a central longitudinal axis thereof in a counterclockwise direction when viewed from the distal portion of the first metatarsal. In order to rotate the first metatarsal, the physician may grasp the first stabilization wire and apply a force thereto thus implementing the first stabilization wire as a lever with the force driving rotation of the first metatarsal. In some aspects, this may be done while the intramedullary angle is being reduced as described in the step 3022. In some aspects, the physician may perform the step 3020 with one hand while maintaining the desired intramedullary correction achieved in the step 3022. The first stabilization wire may also be manipulated by the physician through the implementation of a tool (e.g., a component that aids the grip of and/or application of force to the first stabilization wire).

The process 3000 is shown to include a step 3022 reducing an intramedullary angle between the first and second metatarsals, according to some aspects. It should be noted that in many surgical procedures, the steps 3020 and 3022 may be performed simultaneously and, in some aspects, it may even be advantageous for a physician to performs these two steps simultaneously. The step 3022 may include manipulating the handle portion of the clamp (e.g., the handle portion 510 including at least one of handle elements 512 and 514 of the clamp 500) such that the distal portion of the first metatarsal is moved closer to the second metatarsal (e.g., moved laterally by the interface portion of the first metatarsal arm applying a force in the lateral direction) thus reducing the intramedullary angle formed between longitudinal axes running along the first and second metatarsals. In some aspects, the second metatarsal may also be manipulated. In some aspects, the second metatarsal may be held static with the first metatarsal being the only metatarsal that is repositioned.

The process 3000 is shown to include a step 3024 inserting a second stabilization wire through a through hole in the clamp and into the first metatarsal via the third incision, according to some aspects. In some aspects, the second stabilization wire may be inserted percutaneously in instances in which a third incision was not made. The second stabilization wire of the step 3024 may be the same as or similar to the stabilization wire 529 as shown and described previously with reference to the clamp 500. The position of the interface portion in the step 3016 is conducive to performing the step 3024 in that the opening 528 is positioned near the third incision such that the physician may insert the second stabilization wire into and through the opening of the interface portion (e.g., the same as or similar to the opening 528) and into the first metatarsal. Once coupled with the first metatarsal of the patient and engaged with the clamp via the opening of the interface portion, the second stabilization wire may be retained in a desired position by the features of the clamp. Further, in some aspects the second stabilization wire may be an olive wire (in some cases, a threaded olive wire) where the olive wire includes a depth stop (e.g., a portion on the shaft of the wire with a diameter greater than that of the opening in the interface portion of the clamp.

The process 3000 is shown to include a step 3026 releasably coupling one or more guide instruments to the retention element, according to some aspects. The guides of the step 3026 may include those shown and described in FIGS. 23-26 or may also include other alternate cut guides. The guides of the step 3026 may be configured to releasably couple with the cap portion and/or other portions of the retention element such that the guides may be coupled and placed while the clamp remains engaged with the first and second metatarsals. The guide instruments may include guides configured to guide one or more cuts to the medial cuneiform, where the guide instruments reference the second metatarsal and/or other adjacent anatomical structures. In some aspects, the guide instruments may be configured to guide a cut to the distal surface of the medial cuneiform that is substantially parallel to the surface of the proximal portion of the first metatarsal after a cut was made previously in the process 3000 (to prepare and release the joint).

The process 3000 is shown to include a step 3028 performing one or more cuts to a medial cuneiform of the patient and prepare the joint space for fusion, according to some aspects. As mentioned in the step 3026, the cut of the step 3028 may be guided by the guide instruments such that the cut to the medial cuneiform such that the resulting distal surface of the medial cuneiform is substantially parallel to the already prepared proximal surface of the first metatarsal. The cut of the step 3026 may be made using a reciprocating saw or other similar instrument commonly used in making similar cuts in similar surgical procedures. After performing the cut of the step 3028, the guide instruments may be decoupled from the cap portion and/or retention element. Further, the joint space of the Lapidus joint may be prepared for fusion (e.g., fenestrate surfaces, etc.).

The process 3000 is shown to include a step 3030 applying a fusion device across the Lapidus joint, according to some aspects. The step 3030 also includes the positioning of the first metatarsal and the medial cuneiform in a desired orientation for fusion. In order to achieve this orientation, the physician may dorsiflex the hallux (e.g., “big toe”) to engage the Windlass Mechanism, with the hallux disposed between the arms of the clamp (where the arms of the clamp are designed so as to leave a space for the hallux to be dorsiflexed). The physician may then place a third and fourth stabilization wire across the joint to stabilize the joint. Once these third and fourth stabilization wires are placed, a fixation device (e.g., plate, fusion device, etc.) is applied across the joint and coupled with both the first metatarsal and the medial cuneiform. Once the fixation device has been fixed, the clamp may be removed (including the second stabilization wire) and the incisions closed.

It should be noted that the steps of the process 3000 may be performed in alternate orders, may have steps added, steps omitted, steps repeated, or be otherwise modified in performing a Lapidus procedure as shown and described herein. For example, prior to fixing the fixation device to the first metatarsal and first cuneiform the physician may manipulate surrounding structures to ensure that the structured maintain function, structural stability, and otherwise provide necessary balance for the patient. Similar, in some aspects one or more cuts may be made to the first metatarsal and/or medial cuneiform. For example, the physician may make a first cut and determine that a greater amount of the bone needs to be resected, thus requiring a second cut be made using either the same or a second guide instrument. In another exemplary procedure, the process 3000 may be modified such that a physician performs cuts to the first metatarsal and the medial cuneiform (the same as or similar to the cuts described previously herein) prior to the application of any surgical clamp (e.g., those shown and described herein). The cuts to the first metatarsal and the medial cuneiform may be made using one or more cut guides such as those shown and described herein (or other similar cut guides), where the cut guides are angled and guide the cuts to achieve a desired correction. The application of the surgical clamp may be implemented (and the clamp manipulated as described herein) to achieve desired correction of the joint, with a fixation device (e.g., intramedullary nail, plating, etc.) applied prior to the removal of the surgical clamp.

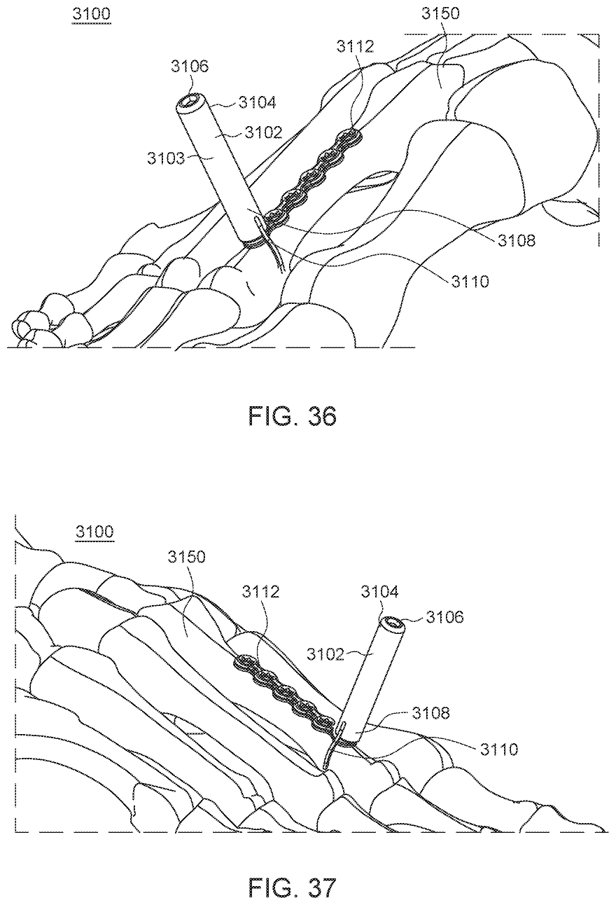

Referring now to FIGS. 36-38, a system 3100 for positioning an orthopedic implant is shown, in accordance with the present disclosure. The system 3100 is shown to include a placement mechanism 3102 configured to facilitate placement of one or more orthopedic implants (e.g., plates, intramedullary nails, other orthopedic implants and/or fixation devices, etc.), for example an implant 3112 as shown. The placement mechanism 3102 may be configured to releasably or otherwise couple with the implant 3112 so as to facilitate insertion through an opening in the skin and/or intraoperative manipulation of the implant 3112 and/or portions of the anatomy. In some aspects, the system 3100 may be configured to accommodate implants for specific portions of the anatomy (e.g., the placement mechanism 3102 may have one or more features/components that facilitates coupling with specific implants for specific anatomy). The system 3100 may also include additional components to those shown in the exemplary embodiments of FIGS. 36-38, for example fasteners (e.g., screws, etc.), alignment guides, drills/drill bits, and other components common to orthopedic systems. Similarly, the system 3100 (or one or more components thereof) may also be implemented in conjunction with other systems. For example, the placement mechanism 3102 may be implemented with various implants, alignment systems, or other hardware or software components.

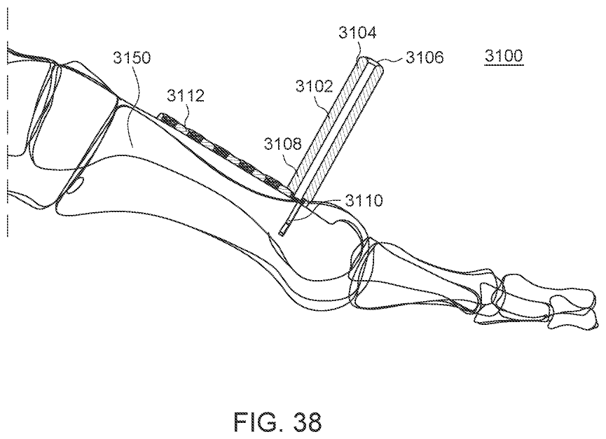

The placement mechanism 3102 is shown to include a body 3103 having a substantially cylindrical shape, although the body 3103 may have alternate geometries and dimensions in alternate embodiments. In some aspects, the body may include a textured outer surface or an alternate geometry of the outer surface (e.g., texture to facilitate gripping or outer surface geometry with ergonomic features). The body 3103 is shown to include a cannulation as shown in the cross-sectional view of FIG. 38, with the cannulation centered about a longitudinal axis of the body 3103 and extending from a first end 3104 of the body to a second end 3108 of the body. The cannulation of the body 3103 terminates at an opening 3106 of the first end 3104 and similarly terminates at an opening of the second end 3108 (not shown). In some aspects, the body 3103 may be substantially symmetrical, both cross-sectionally about the longitudinal axis of the body 3103 and/or about a midline disposed between the first and second ends 3104, 3108 running perpendicular to the cannulation of the body 3103. The shape and dimension of the cannulation may vary according to some embodiments of the system 3100. For example, the cannulation of the body 3103 may be configured to accommodate one or more other components such as fixation/stabilization wires, fasteners, drills and/or drill bits, alignment guides, or other instruments and components common to orthopedic procedures. Further, the placement mechanism 3102 may be manipulatable about one or more components disposed at least partially within the cannulation of the body 3103. For example, a stabilization wire may be placed within the cannulation of the body 3103 (and extending through a portion of the implant 3112) and coupled with at least a portion of a bone such that the placement mechanism may be decoupled from any implant (if coupled with any such implant) and withdrawn over the stabilization wire. In some aspects, the system 3100 may include components sized such that the components may be received by and manipulatable within the cannulation of the body 3103.

The placement mechanism 3102 is further shown to include a coupling mechanism 3110 extending from the second end 3108 of the body 3103. As shown, the coupling mechanism 3110 includes a pair of extensions arranged substantially diametrically opposite on another at the second end 3108 of the body 3103, with each of the extensions extending from the outer surface of the second end 3108. It should be understood that alternate embodiments of the system 3100 and/or any components thereof may include alternate coupling mechanisms similar to the coupling mechanism 3110 as shown. For example, the coupling mechanism 3100 may include additional or fewer extensions, extensions arranged in an alternate configuration to that of the exemplary embodiment of FIGS. 36-38, or extensions with alternate geometries (e.g., greater/lesser lengths, curvatures, radii of curvature, etc.). In some aspects, the coupling mechanism 3110 (including some or all of any extensions such as those shown and described herein, or any other components) may otherwise extend from the body 3108. For example, the coupling mechanism 3110 and any components thereof (e.g., extensions) may be integral with the body 3103 or may extend from the second end 3108 adjacent the opening at the second end 3108 that is a terminal end of the cannulation of the body 3103.

As shown in FIGS. 36-38, the system 3100 includes the placement mechanism 3102 releasably coupled with a second metatarsal 3150 via the coupling mechanism 3110. The system 3100 further includes an implant 3112, which is shown as a plate in the exemplary embodiments of FIGS. 36-38 and is disposed between the second end 3108 of the placement mechanism 3102 and the superior surface of the second metatarsal 3150. The implant 3112 is intended to be exemplary, as the system 3100 may include various implants of alternate sizes and/or geometries. Similarly, the system 3100 and components thereof may be implemented in procedures with portions of the anatomy other than the second metatarsal (e.g., other bones of the foot, ankle, hand, wrist, etc.). For example, the placement mechanism 3102 and components thereof may have alternate geometries in order to couple with larger/smaller implants and portions of the anatomy (e.g., longer coupling mechanisms, etc.). In some aspects, the implant 3112 may be releasably coupled with the placement mechanism 3102 via one or more coupling features (for example, the geometry of the coupling mechanism 3110 may retain the implant 3112 adjacent to the second end 3108 of the placement mechanism 3102 via one or more of a notch, compression fit, or other known coupling configuration). The placement mechanism 3102 (with or without the implant 3112 releasably coupled therewith) may be releasably coupled with the second metatarsal 3150 from a substantially dorsal direction (as shown), where the placement mechanism 3102 is pushed down onto the superior surface of the second metatarsal 3150. For alternate embodiments and alternate geometries, the placement mechanism 3102 may be otherwise positioned, placed, and/or coupled. In some aspects, the coupling mechanism 3110 (and any components thereof) may accommodate the second metatarsal 3150 so as to releasably couple with the second metatarsal 3150. In some aspects, the coupling mechanism 3110 (and components thereof) may be pliable or otherwise manipulatable with a geometric configuration such that pushing the placement mechanism 3102 and thus, the coupling mechanism 3110 down on the superior surface of the second metatarsal 3150 manipulates the coupling mechanism 3110 so as to extend at least partially around the second metatarsal 3150.

The placement mechanism 3102 may be gripped by a physician on the body portion 3103 and be manipulated relative to the second metatarsal 3150. For example, the physician may manipulate the placement mechanism 3102 similar to a joystick, with the placement mechanism 3102 and the implant 3112 translatable about the surface of the second metatarsal 3150 (e.g., distal, proximal, medial, lateral movements so as to reposition the placement member 3102 and implant 3112 about the surface of the second metatarsal 3150). Once a desired position has been reached, the physician may insert a stabilization wire through the opening 3106, into the cannulation of the body 3013, through an opening in the implant 3100, and into the second metatarsal 3150 so as to retain the placement mechanism 3102 and the implant 3112 relative to the second metatarsal 3150. Once the stabilization wire is placed, the physician may again manipulate the placement mechanism 3102 (similar to how a joystick is used) so as to manipulate the second metatarsal 3150 (as the system 3100 is now coupled with the second metatarsal 3150). The physician may then remove the placement mechanism 3102 over the stabilization wire (thus decoupling the placement mechanism 3102 with the second metatarsal 3150), or the physician may implement additional surgical instrumentation (e.g., cannulated drill, fasteners, etc.) over the stabilization wire and within the cannulation of the body 3103 so as to further the ongoing procedure. Ultimately, the stabilization wire will be removed prior to the end of the procedure. This process may be repeated multiple times with various instrumentation and components. As shown, the placement mechanism 3102 is coupled with an end portion of the implant 3112 adjacent to an end opening in the implant 3100. However, the placement mechanism 3102 may be releasably couplable with the implant 3112 at various points along the length of the implant 3112, for example at each opening of the implant 3112. Accordingly, a physician may releasably couple and decouple the placement mechanism 3102 with the implant 3112 multiple times at multiple positions along the implant 3112, with surgical steps the same as or similar to those described previously performed at each respective coupling position. In some aspects, a physician may implement multiple systems 3100 and/or components thereof in a single procedure, for example two placement mechanisms 3102 simultaneously coupled with a single implant 3112 or two systems 3100 with each releasably coupled with different bones or portions of the bone.

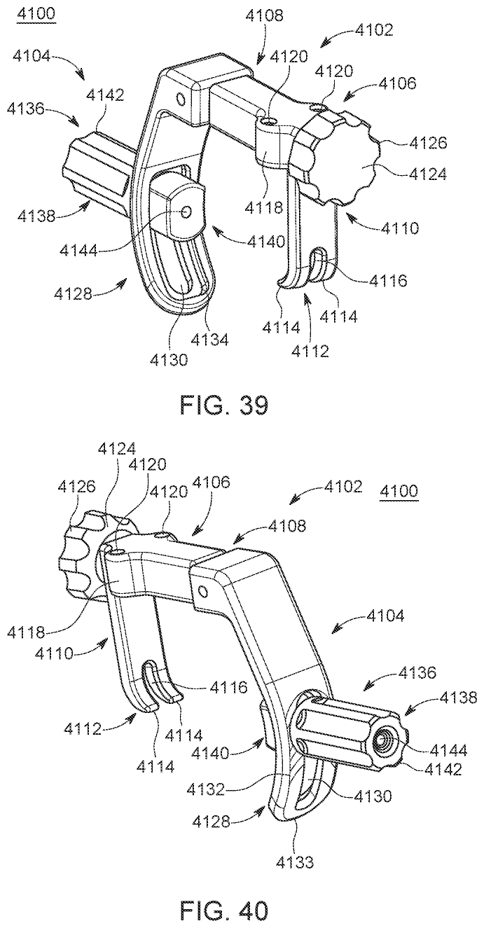

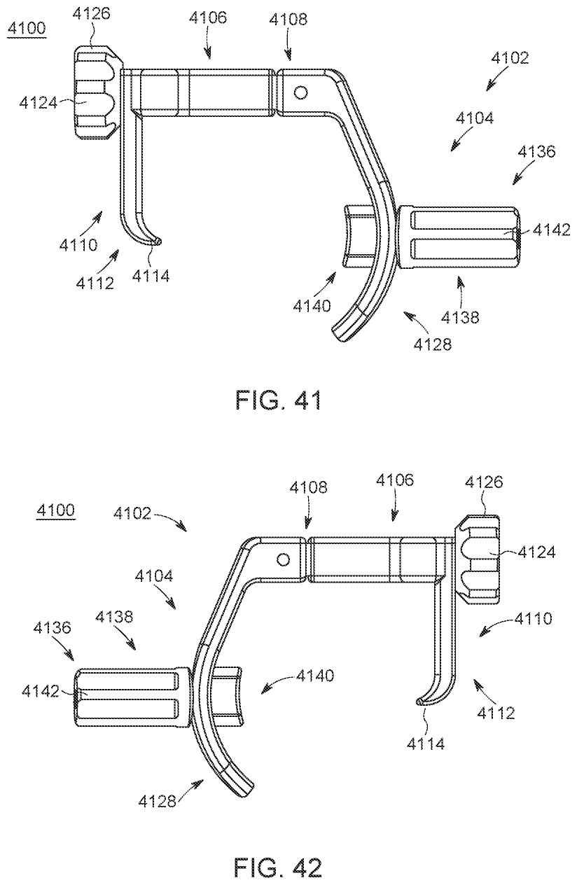

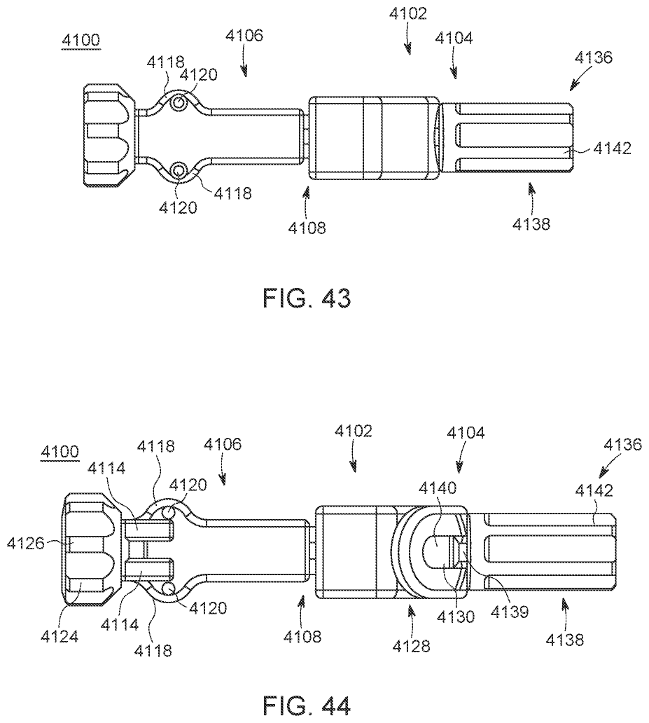

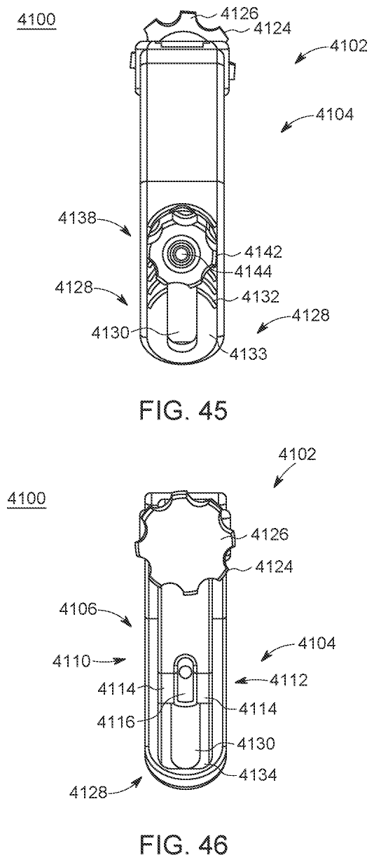

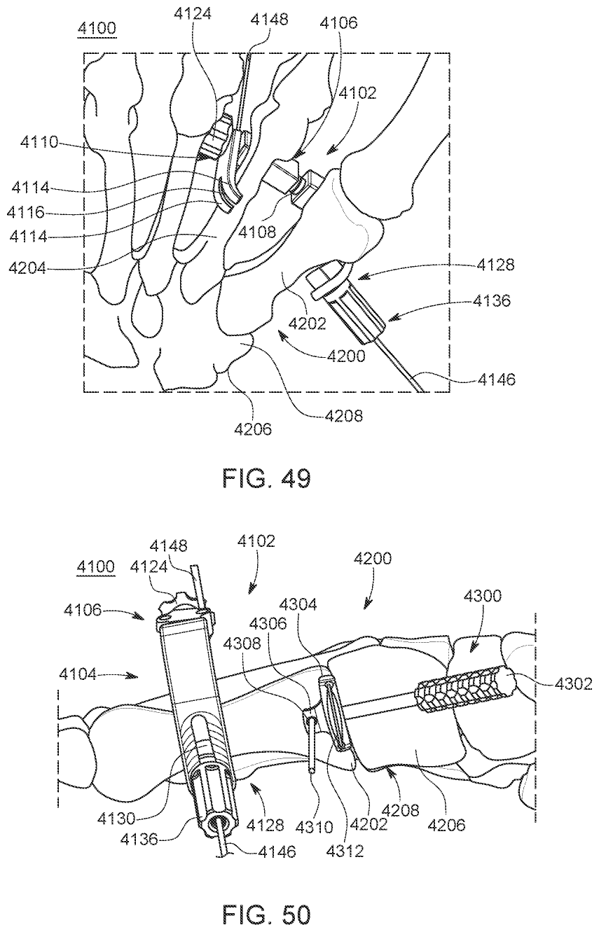

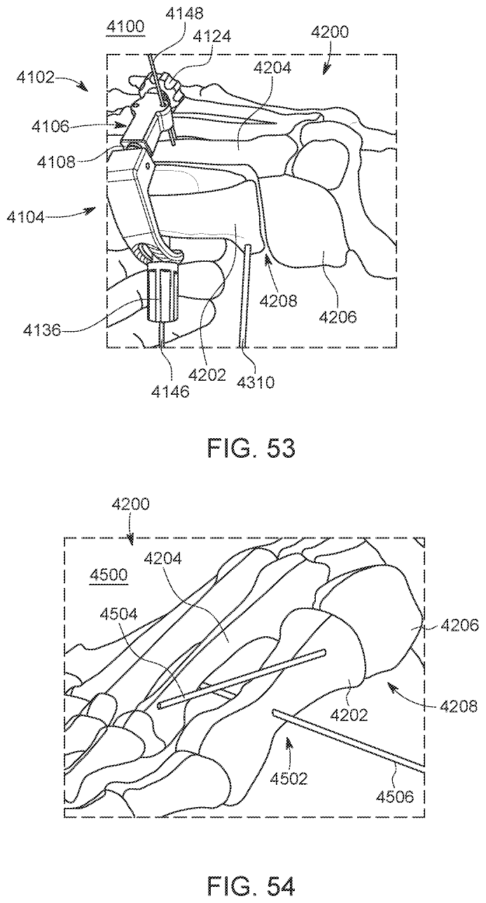

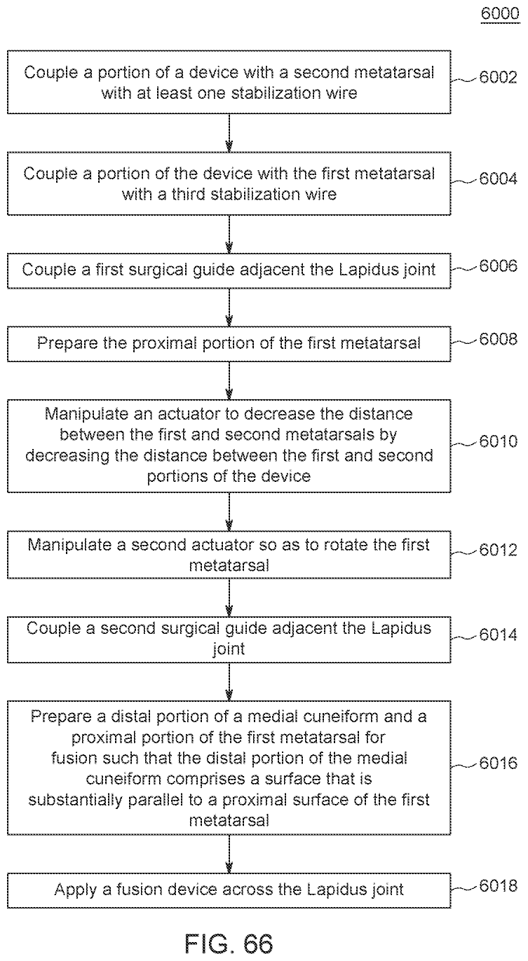

Referring to FIGS. 39-54, there is illustrated an exemplary embodiment of a system 4100 for implementation in performing a procedure on one or more anatomical structures of the foot (for example, procedures of the Lapidus joint such as arthrodesis or other similar fusion procedures). The system 4100, as shown and described herein with reference to FIGS. 39-54, includes a clamp 4102 and may further include one or more additional components, for example one or more surgical guides such as those shown and described herein. Additionally, the system 4100 may also be implemented in performing one or more steps of the methods shown and described with reference to FIG. 66. Further, it should be understood that the system 4100 may include one or more components in addition to those shown and described herein (e.g., cutting instruments, saws, scalpels, forceps, retractors, etc.) and accordingly, the method shown and described with reference to FIG. 66 may incorporate the additional components.

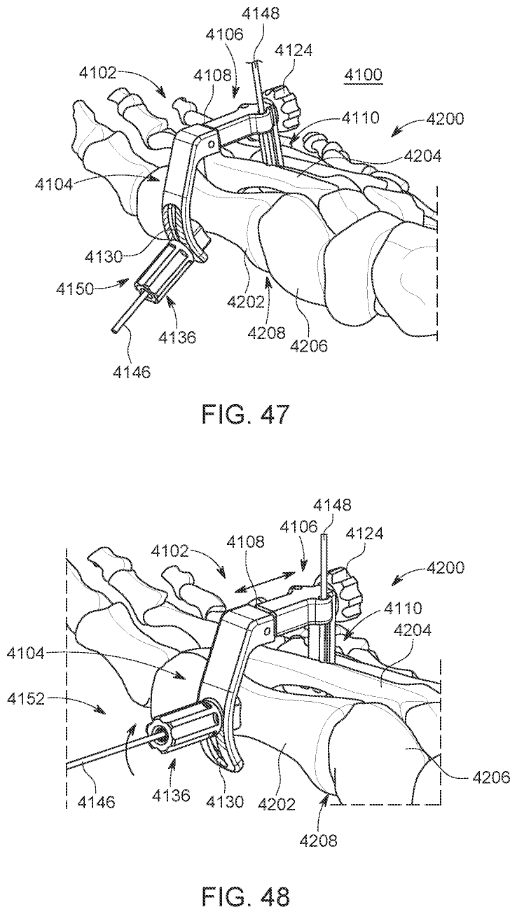

FIGS. 39-54 illustrate an exemplary instrument, shown as the clamp 4102, for determining, aligning/realigning, correcting, positioning/repositioning, rotating/derotating, or otherwise manipulating one or more anatomical structures of the foot relative to other anatomical structures of the foot in accordance with the present disclosure. As shown and described herein, the clamp 4102 is configured to releasably couple with structures of a foot 4200 shown and described in FIGS. 47-53 (shown as reference numeral 4200) including a first metatarsal (shown as reference numeral 4202) and a second metatarsal (shown as reference numeral 4204) so as to manipulate a Lapidus joint (shown as reference numeral 4208) configured between the first metatarsal 4204 and a medial cuneiform (shown as reference numeral 4206). In some aspects, the clamp 4102 may be implemented on distal portions of the first and second metatarsals 4202, 4204, but may also be implemented elsewhere about the first and second metatarsals 4202, 4204. The clamp (and components thereof) 4102 is configured to facilitate the correction of the IM angle formed between a first extended longitudinal axis of the first metatarsal 4202 and a second extended longitudinal axis of the second metatarsal 4204. Further, the clamp 4102 is configured to facilitate derotation (e.g., rotation opposite that which caused a bunion or other deformity) of the first metatarsal 4202 relative to the second metatarsal 4204. Correction of the IM angle and/or derotation of the first metatarsal 4202 relative to the second metatarsal 4204 may be performed by a physician in a procedure to address a deformity at or near the Lapidus joint (e.g., bunion, arthrodesis of Lapidus joint, etc.).

The clamp 4102 is shown to include a first portion 4104 and a second portion 4106, where the first portion 4104 is releasably coupled with the second portion 4106 by/at a coupling 4108. In some aspects, the coupling 4108 may include a coupling mechanism (e.g., a screw and complimentary bore, etc.) where a first component of the coupling is coupled or integral with the first portion 4104 and a second component complimentary to the first component is coupled with or integral with the second portion 4106. Further, in some aspects the coupling 4108 may include a portion of a coupling mechanism, where at least a portion of the coupling mechanism is configured to extend at least partially within the second portion 4106. Such a portion of a coupling mechanism may be coupled with or integral with the first portion 4104 and further may extend at least partially into an opening of the second portion 4106. In some aspects, at least a portion of the coupling may include incremental markers (e.g., indicators, measurement markers, etc.) to indicate expansion of the clamp 4102. The coupling 4108 (and any components thereof) may also facilitate motion of the first portion 4104 relative to the second portion 4106, or facilitate motion of the second portion 4106 relative to the first portion 4104. In some aspects, an actuator 4124 (e.g., knob, etc.) positioned substantially along a longitudinal axis of the second portion 4106 may be configured to releasably couple with a portion of the second portion 4106 of the clamp 4102. For example, the actuator 4124 may include a protrusion (e.g., male component with threading, set screw, etc.) that extends longitudinally into and through a bore disposed in the second portion 4106 of the clamp and facilitates releasable coupling at the coupling 4108 with the first portion of the clamp 4104 (e.g., complimentary threads, etc.). In some aspects, the bore may include a coupling mechanism therein (e.g., threading, etc.) configured to releasably couple with a complimentary portion of the actuator 4124 (e.g., the protrusion which may have a coupling mechanism such as threading, etc.). Accordingly, manipulation of the actuator 4124 via a texture 4126 (e.g., to facilitate grip, rotation, manipulation, etc.) may translate the second portion 4106 toward or away from the first portion 4104 thus expanding the clamp 4102. In some aspects, the translation may be along a longitudinal axis that is shared by (e.g., common) one or more of the actuator 4124, the protrusion from the actuator 4124, the bore of the second portion 4106, and any complimentary coupling mechanisms of the first portion 4104. Further, the first portion 4104 and the second portion 4106 may be coupled such that one or both portions may be manipulatable about the longitudinal axis (e.g., the shared/common axis).

The second portion 4106 of the clamp 4102 is shown to include a pair of protuberances 4118 extending laterally from the second portion 4106. As shown in FIGS. 39-54, the protuberances 4118 are disposed along the second portion 4106 such that the pair of protuberances are positioned an equal distance from the coupling 4108 and are further positioned closer the actuator 4124 than the coupling 4108. In some aspects, the actuator 4124 may include at least a portion of a coupling mechanism configured complimentary to that of the coupling 4108. For example, the actuator 4124 may include an extension having a threading configured to be received within at least a portion of the coupling 4108. In some aspects, the extension of the actuator 4124 may be configured to releasably couple the second portion 4106 with the first portion 4104 via the coupling 4108. The protuberances 4118 are shown to be substantially hemispherical, although the protuberances 4118 may include alternate geometries in some aspects of the clamp 4102. Each of the protuberances is shown to include a bore 4120 having a substantially cylindrical shape and extending from a top surface through to a bottom surface of the second portion 4106 so as to establish fluid communication therebetween. The bores 4120 may be configured to receive one or more common surgical instruments, for example stabilization wires, k-wires, olive wires, and/or other stabilization elements so as to releasably couple the second portion 4106 with the second metatarsal 4204.

As shown in FIGS. 47-53, the stabilization wires may be placed such that the wires are received by the bores 4120, with the wires inserted into a substantially dorsal portion of the second metatarsal 4204 (and as shown in FIGS. 47-53, positioned nearer the proximal end of the second metatarsal 4204 than the distal end). For example, as shown in FIG. 50, one of the bores 4120 is shown to have received a second stabilization wire 4148, with the second of the two bores 4120 vacant. In some aspects, a physician may elect to place a stabilization wire (such as the second stabilization wire 4148) in one/either of the bores 4120 rather than placing a stabilization wire in each of the bores 4120. When releasably coupled with the second metatarsal 4204 via stabilization wires received through the bores 4120, the second portion 4106 of the clamp 4102 may be configured in a static position should the actuator 4124 be manipulated so as to translate the first portion 4104 toward the second portion 4106 (e.g., the second portion 4106 fixed to the second metatarsal 4204 would be static while manipulation of the actuator 4124 would translate the first portion 4104 toward the second portion 4106 and the second metatarsal 4204). In some aspects, the bores 4120 may be of different sizes (e.g., both larger, both smaller, or one larger/one smaller) and/or geometries (e.g., rectangular, triangular, etc.) so as to accommodate the corresponding geometries of various stabilization elements. Each of the pair of protuberances 4118 is shown to have an equal size, although in some aspects, the second portion 4106 of the clamp 4102 may include one, two, or more protuberances that are the same as or similar to the protuberances 4118 (having equal or unequal sizing) disposed variously along the second portion 4106. Further, in some aspects, one or more of the protuberances 4118 may include zero, one, two, or more than two bores arranged various about the protuberances 4118. As shown, each of the bores 4120 are positioned substantially equidistant from the outer edge of the protuberances 4118, although the bores 4120 may be positioned alternately in some aspects of the clamp 4102.

The second portion 4106 of the clamp 4102 is shown to include an extension 4110 configured opposite the second portion 4106 from the coupling 4108 and, as shown in FIGS. 39-54, extending from the second portion 4106 from a point adjacent the actuator 4124 in a substantial orthogonal angle from the second portion 4106. As shown, the extension 4110 has a substantially rectangular cross-sectional geometry, but may have alternate geometries in some aspects of the system 4100. The extension 4110 includes a retention mechanism 4114, shown in FIGS. 39-54 as a pair of protrusions disposed at a distal end of the extension 4110 (e.g., opposite the second portion 4106 adjacent the actuator 4124) defining a cavity 4116 disposed substantially between the pair of protrusions of the retention mechanism 4114. As shown, the retention mechanism 4114 is integral to the extension 4110 and has a curvature substantially toward the first portion 4104 of the clamp 4102 (e.g., adjacent to a tangent line of the curvature that is substantially perpendicular to the second portion 4106) with the distal-most portions of the retention mechanism 4114 pointing substantially toward the first portion 4104 of the clamp 4102. The cavity 4116 is shown to have a substantially hemi-elliptical geometry, with the hemi-elliptical geometry disposed at proximal-most portion of the cavity 4116. However, in some aspects, the cavity 4116 (as well as the retention mechanism 4114) may have alternate thicknesses/widths, lengths, depths, curvatures, and other geometric properties.

The extension 4110 and the retention mechanism 4114, as shown in FIGS. 47-53, are configured to at least partially interface with (e.g., engage with, contact, etc.) a flat of the second metatarsal 4204 positioned on a substantially lateral surface of the second metatarsal 4104. The retention mechanism 4114, including the distal-most portion thereof may be configured to contact a substantially inferior portion of the flat on the lateral surface of the second metatarsal 4204. As shown, the curvature of the distal-most portion of the retention mechanism 4114 is configured to at least partially surround the second metatarsal 4204 such that the retention mechanism 4114 contacts at least a portion of the plantar surface of the second metatarsal 4204 (and thus engages multiple surfaces of the second metatarsal 4204). The curvature of the retention mechanism 4114 thus accommodates the geometry of the second metatarsal 4204 so as to maximize the engagement of the retention mechanism 4114 with the second metatarsal 4204 should a force be applied to the second metatarsal 4204 via the retention mechanism 4114 (e.g., on a lateral and/or plantar surface of the second metatarsal 4204 and applied in a medial and/or superior direction). Accordingly, the retention mechanism 4114 maximizes the engagement of the extension 4110 of the second portion 4106 of the clamp 4102 with the second metatarsal 4204 so as to reduce/prevent loss of contact/engagement of the clamp 4102 with the second metatarsal 4204 as one or more forces are applied to the second metatarsal 4204 (e.g., manipulation of the actuator 4124 so as to translate one or both of the first portion 4104 and the second portion 4106 toward or away from one another while the extension 4110 and/or the retention mechanism 4114 are in contact with the second metatarsal 4204). In some aspects, the cavity 4116 may be configured to receive (e.g., permit passthrough) one or more stabilization wires.

The first portion 4104 of the clamp 4102 is shown to have a substantially curved geometry (e.g., convex), with the curvature of the first portion 4104 pointing substantially in the direction of the second portion 4106. The first portion 4104 includes an aperture 4130 disposed substantially on a distal portion 4128 (e.g., distal half) of the first portion 4104. The aperture 4130 as shown in FIGS. 39-54 has an elongated, rounded geometry (e.g., elliptical, oval, etc.) with the aperture 4130 having the same curvature as the first portion 4104. The aperture 4130 is disposed within an inner depression 4133 on the concave surface and an outer depression 4134 on the convex surface of the first portion 4104. The inner depression 4133 and the outer depression 4134 are shown to have similar depth, length, and width dimensions, with the inner depression 4133 and the outer depression 4134 having a greater length and width than the aperture 4130 in the same/a similar geometric shape. Further, the inner depression 4133 and the outer depression 4134 are positioned in approximately the same location on their respective surfaces of the distal portion 4128 of the first portion 4104.

The first portion 4104 is further shown to include an actuator 4136 having a first portion 4138 and a second portion 4140, with the first portion 4138 and the second portion 4140 separated by a connector 4139. The connector 4139 is shown to have a substantially lesser cross-sectional geometry than that of the first portion 4138 or the second portion 4140. In some aspects, the connector 4139 may include an extension having at least a portion of a coupling mechanism (e.g., a threading) configured to be complimentary to an opening disposed substantially along a longitudinal axis of the first portion 4138. According, such a coupling mechanism may facilitate releasably coupling between the first and second portions 4138, 4140 of the actuator 4136. As shown in FIGS. 39-54, the connector 4139 has a substantially cylindrical geometry with a diameter/lateral dimension slightly lesser than that of the aperture 4130. As seen in FIGS. 39-54, the first portion 4138 is shown to have a substantially cylindrical shape with a diameter slightly lesser than the width of the outer depression 4134. The second portion 4140 is shown to also have a lateral dimension slightly less than the width of the inner depression 4133. FIGS. 39-54 show the actuator 4136 to be positioned such that at least a portion of the connector 4139 is disposed within the aperture 4130. Similarly, a distal-most portion of the first portion 4140 is disposed within the outer depression 4134 and the proximal-most portion of the second portion 4140 is disposed within the inner depression 4133. Accordingly, the range of movement of the actuator 4136 is defined by the dimensions of the aperture 4130 (and in some aspects, also the inner depression 4133 and the outer depression 4134). The actuator 4136 may be manipulated through a defined range of motion along the curvature of the first portion 4104, with the bounds of the range of motion defined by the aperture 4130 (and, in some aspects the inner depression 4133 and/or the outer depression 4134). The actuator 4136 also includes a texture 4142 on an outer surface of the first portion 4138 of the actuator 4136, with the texture configured to facilitate gripping and/or manipulating the actuator 4136 (e.g., through the range of motion, rotation, etc.).

The actuator 4136 includes a bore 4144 extending from the first portion 4138, through the connector 4139 and to the second portion 4140 along a longitudinal axis of the actuator 4136 such that fluid communication is established through the length of the actuator 4136. As shown in FIGS. 39-54, the bore 4144 has a substantially cylindrical geometry configured to receive a stabilization wire (e.g., a k-wire, olive wire, etc.) that may be passed into and through the bore 4144 of the actuator 4136. As shown in FIGS. 47-53, a first stabilization wire 4148 may be received by the bore 4144, and inserted into (e.g., releasably coupled with) the first metatarsal 4202 (as shown, a substantially medial portion of the first metatarsal 4202. Accordingly, when the first stabilization wire 4146 is placed in the first metatarsal 4202, the actuator 4136 is releasably coupled with the first metatarsal 4202. By manipulating the actuator 4136 within the range of motion defined by the movement of the connector 4139 within the aperture 4130, the first metatarsal 4202 is moved accordingly.

FIG. 47 shows the actuator 4136 as being releasably coupled with the first metatarsal 4202 via the first stabilization wire 4146 with the actuator 4136 in a first position 4150. The actuator 4136 is coupled such that the distal-most surface of the second portion 4140 of the actuator 4136 is positioned at or near a surface of the first metatarsal 4202 (as shown, a substantially medial surface). In the first position 4150, the connector 4139 of the actuator 4136 is shown to be at an inferior-most position within the aperture 4130 such that a portion of the connector 4109 abuts an edge of the inner depression 4133 and the outer depression 4134 (and, accordingly, the first portion 4138 of the actuator 4136 is at an inferior-most portion of the outer depression 4134 and the second portion 4140 of the actuator 4136 is at an inferior-most portion of the inner depression 4133). Manipulation of the actuator 4136 in a substantially superior direction from the first position 4150 to a second position 4152 (as shown in FIG. 48) substantially rotates (or derotates) the first metatarsal 4202 about a longitudinal axis thereof. For example, in order to correct a portion of a deformity or other concern with the Lapidus joint 4208 (or components thereof, such as the first metatarsal 4202 having rotated) a physician may manipulate the actuator 4136 from the first position 4150 to the second position 4152, thus rotating/derotating the first metatarsal 4202 in the frontal plane and thereby correcting any rotational deformity of the first metatarsal 4202. In some aspects, the actuator 4136 may be in a third position between the first position 4150 and the second position 4152 when coupled with the first metatarsal 4202 by the first stabilization wire 4146. Similarly, in some aspects the actuator 4136 may be manipulated by a physician from a position such as the aforementioned third position to a fourth position between the third position and the second position 4152. When manipulating the actuator 4136 using the texture 4142, a set of notches 4132 are visible within the outer depression 4134. The notches 4132 indicate incremental movement as the actuator is manipulated about the defined range of motion (e.g., defined by the aperture 4130 and the connector 4139 positioned therein). In some aspects, the notches 4132 may correspond to set increments (e.g., each notch is representative of 5, 10, or 15 degrees of rotation, etc.).

Once the clamp 4102 is secured to the foot 4200 via the first stabilization wire 4146 releasably coupling the actuator 4136 with the first metatarsal 4202 as well as at least the second stabilization wire 4148 (if not multiple second stabilization wires) releasably coupling the second portion 4106 to the second metatarsal 4204, one or both of the actuators 4124, 4136 may be manipulated (e.g., actuated) so as to position/reposition the first and/or second metatarsals 4202, 4204 in one or more anatomical planes. For example, the actuator 4124 may be manipulated (for example, turned as in the knob/set screw configuration shown in the exemplary embodiment of FIGS. 39-53) such that the first portion 4104 is translated along the longitudinal axis of the actuator 4124 in a substantially lateral direction (e.g., toward the second portion 4106, as shown by the double arrow in FIG. 48). Accordingly, with the first portion 4104 coupled with the first metatarsal 4202 via the actuator 4136 and the first stabilization wire 4146, such movement of the first portion 4104 (in the substantially lateral direction, toward the second portion 4106 along the longitudinal axis of the actuator 4124) manipulates the first metatarsal 4202 such that the IM angle between the first metatarsal 4202 and the second metatarsal 4204 (e.g., an angle formed by the longitudinal axes of the first metatarsal 4202 and the second metatarsal 4204) is decreased. As such a force is applied, the clamp 4102 and various components thereof remain releasably coupled with and/or otherwise maintaining contact with one or more portions of the first and second metatarsals 4202, 4204.

As mentioned previously, the first metatarsal 4202 may further be positioned/repositioned by manipulation of the actuator 4136 (once the second portion 4106 has been coupled with the second metatarsal 4204). Manipulation of the actuator 4136 (via the first portion 4138) within the range of motion defined by the aperture 4130 and the connector 4139 disposed therein (e.g., from the first position 4150 in FIG. 47 to the second position 4152 in FIG. 48, such as that shown by the arrow in FIG. 48) applies a rotational force to the first metatarsal 4202 in the frontal plane so as to rotate/derotate the first metatarsal (e.g., to address a rotational deformity). While such a force is applied to the first metatarsal via the actuator 4136, the first portion 4104, the second portion 4106 and other components of the clamp 4102 remain static as a function of the releasable coupling with the first metatarsal 4202 and the second metatarsal 4204 (e.g., the second stabilization wire(s) 4148 releasably coupling the second portion 4106 with the second metatarsal 4202, the retention mechanism 4114 in contact with at least one surface of the second metatarsal 4202, the releasable coupling of the actuator 4136 with the first metatarsal 4202 via the first stabilization wire 4146, etc.).

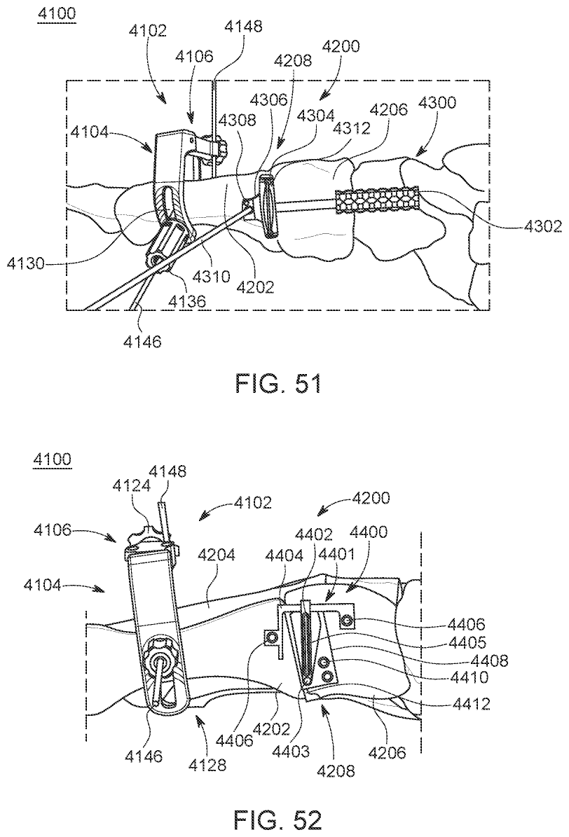

Referring to FIGS. 50-51, a surgical guide 4300 is shown to be coupled with a proximal portion of the first metatarsal 4202 adjacent the Lapidus joint 4208. In some aspects, the surgical guide 4300 may be provided in a kit or other system along with the system 4100 and/or one or more implants and/or fixation components (as well as various other instruments/components). Further, in some aspects, the surgical guide 4300 may be configured to facilitate one or more cuts made to the first metatarsal 4202. The surgical guide 4300 includes a handle 4302 extending laterally from a cut guide 4304 of the surgical guide 4300. As shown, an end portion of the handle 4302 includes a tactile finish configured to facilitate grip of the handle 4302 of the surgical guide 4300. The cut guide 4304 includes a slot 4312 positioned in a central portion of the cut guide 4302. FIGS. 50-51 show the slot 4312 as having a substantially elongated geometry configured to receive one or more cutting instruments (e.g., reciprocating saw, etc.). In some aspects, the slot 4312 may have alternate geometries and/or dimensions, for example the slot 4312 may have greater or lesser length and/or width dimensions than those of the slot as shown. The cut guide 4304 is further shown to include a protuberance 4306 extending laterally from a side of the cut guide 4304 opposite that from which the handle 4302 extends. The protuberance 4306 includes a bore 4308 substantially centered within the top surface of the protuberance 4306, with the bore 4308 extending from the top surface of the protuberance 4306 through to a bottom surface thereof and thus establishing fluid communication between the top and bottom surfaces of the protuberance 4306. The bore 4308 is shown to receive a stabilization wire 4310 (which may be the same as and/or similar to the first and/or second stabilization wires 4146, 4148) therethrough such that the stabilization wire 4310 may releasably couple the surgical guide 4300 with the proximal portion of the first metatarsal 4202. In some aspects, for example, the exemplary surgical guide 4300 shown in FIG. 51, the protuberance 4306 may include a plurality of bores 4308, for example a pair of bores 4308 as shown. In some aspects, one or more stabilization wires 4310 may be received therethrough the bores 4308 to releasably couple the surgical guide 4300 with the first metatarsal 4202 at one or more points. Further, in some aspects, the surgical guide 4300 may include a protrusion (e.g., a paddle, etc., not shown in FIGS. 50-51) configured to be inserted into the joint space of the Lapidus joint 4208 (e.g., between the first metatarsal 4202 and the medial cuneiform 4206).

Referring now to FIG. 52, a surgical guide 4400 is shown to be coupled with a distal portion of the medial cuneiform 4206 and a proximal portion of the first metatarsal adjacent the Lapidus joint 4208. In some aspects, the surgical guide 4400 may be provided in a kit or other system along with the clamp 4100 and/or one or more implants and/or fixation components (as well as various other instruments/components). Further, in some aspects, the surgical guide 4400 may be configured to facilitate one or more cuts made to the medial cuneiform 4206. The surgical guide 4400 includes a body 4401 which is pivotably coupled with a cut guide 4402 at a coupling 4403. The cut guide 4402 includes a slot 4405 positioned in a central portion of the cut guide 4402. As shown in FIG. 52, the slot 4405 has a substantially elongated geometry configured to receive one or more cutting instruments (e.g., reciprocating saw, etc.). In some aspects, the slot 4405 may have alternate geometries and/or dimensions, for example, the slot 405 may have greater or lesser length and/or width dimensions than those of the slot as shown. As shown, the coupling 4403 includes a protrusion of the body 4401 which has a substantially cylindrical geometry and is received by a bore of the cut guide 4402 with a complimentary cylindrical geometry. Accordingly, the coupling 4403 permits pivoting motion of the cut guide 4402 relative to the body 4401. The body 4401 is shown to include a base portion 4408, with the base portion 4408 including a pair of bores 4410. The bores 4410 are configured to receive one or more stabilization wires (one per bore 4410, which may be the same as and/or similar to those shown and described previously herein) so as to facilitate releasable coupling of the surgical guide 4400 with the medial cuneiform 4206. The cut guide 4402 includes a cross member 4404 extending bi-laterally from a distal-most portion of the cut guide 4402. The cross member 4404 comprises a pair of bores 4406 positioned at a distal portion of each segment of the cross member 4404 and are configured to each receive a stabilization wire (such as those shown and described herein). The cut guide 4402 may be pivoted relative to the body 4401 such that one of the bores 4406 is positioned adjacent the medial cuneiform 4206 and the other of the bores is positioned adjacent the first metatarsal 4202. Accordingly, the crossing member 4404 and the bores 4406 thereof are configured to facilitate coupling with the first metatarsal 4202 and/or the medial cuneiform 4206 after being pivoted (with the cut guide 4402) relative to the body 4401. The body further includes a protrusion 4412 configured to be placed within the joint space of the Lapidus joint 4208 (e.g., between the distal portion of the medial cuneiform 4206 and the proximal portion of the first metatarsal 4202).

Referring now to FIG. 54, a fixation system 4500 is shown on the foot 4200. It should be noted that the fixation system 4500 shown in FIG. 54 is in no way limiting, and may include one or more additional fixation system elements including, for example, an intramedullary nail, one or more plates and/or fasteners, drills, etc. The system 4500, shown in part in FIG. 54, includes a pair of wires 4502 including a first wire 4504 and a second wire 4506. The first wire 4504 is shown to be inserted from an anterior-to-posterior direction into a dorsal surface of the proximal portion of the first metatarsal 4202. In some aspects, the first wire 4504 may traverse the Lapidus joint 4208 (or where the Lapidus joint 4208 previously was, if the joint has already been prepared for fusion) and extend into the medial cuneiform 4206. The second wire 4506 is shown to be inserted in a medial-to-lateral direction into a medial surface and out of a lateral surface of the first metatarsal 4202 and into a medial surface of the second metatarsal 4204. In some aspects, the second wire 4506 may extend out of a lateral surface of the second metatarsal 4204. The pair of wires 4502 are configured to provide temporary fixation and/or stability and/or immobility of the first metatarsal 4202, the second metatarsal 4204, the medial cuneiform 4206 and/or other anatomical structures adjacent the Lapidus joint 4208. In some aspects, the pair of wires 4502 may be placed just prior to a fixation element being implanted (e.g., intramedullary nail, one or more plates and complimentary fasteners, etc.).

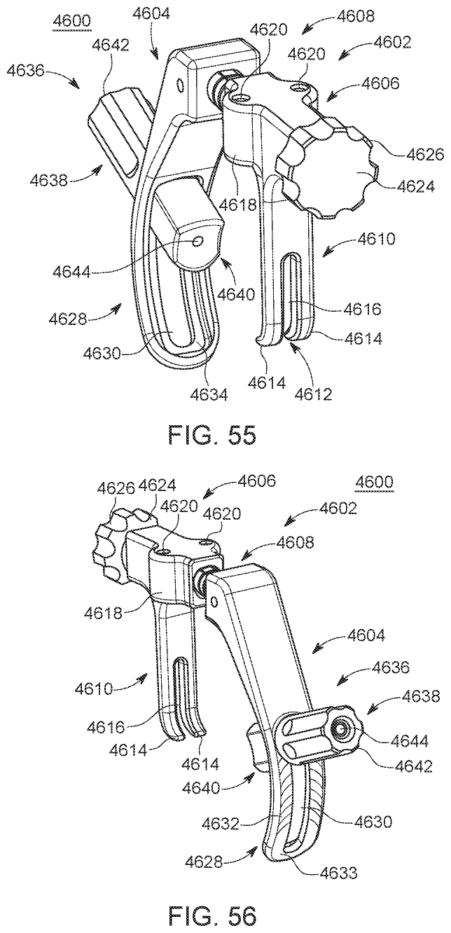

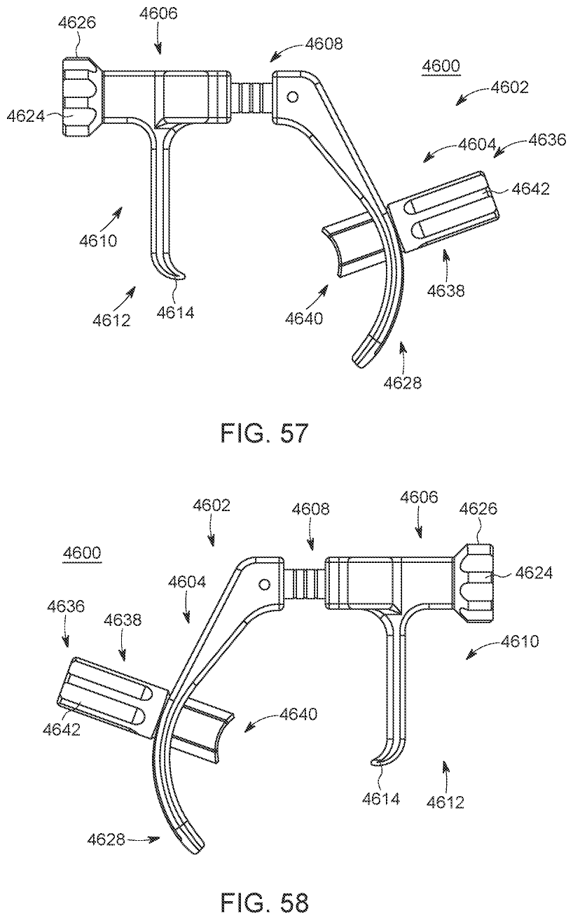

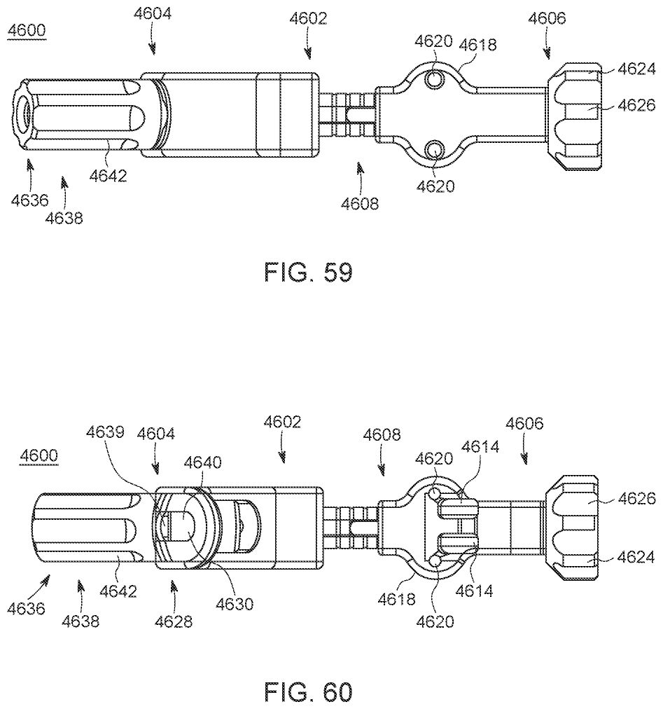

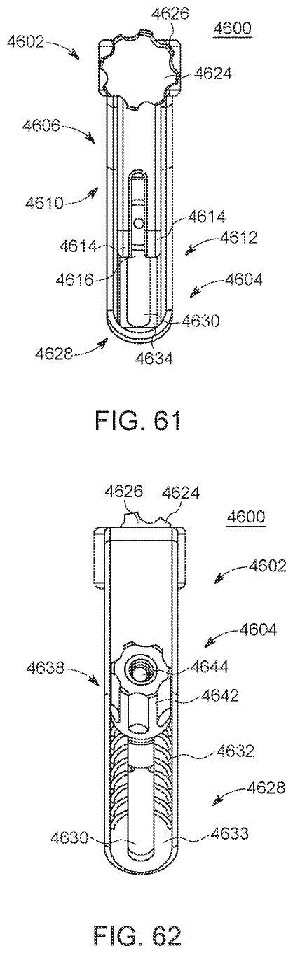

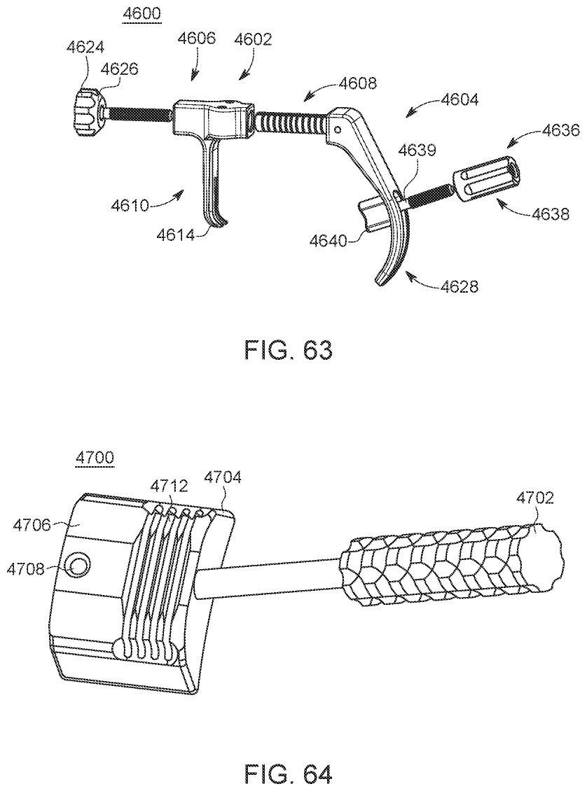

Referring now to FIGS. 55-63, there is illustrated an exemplary embodiment of a system 4600 for implementation in performing a procedure on one or more anatomical structures of the foot (for example, procedures of the Lapidus joint such as arthrodesis or other similar fusion procedures). The system 4600 may include one or more components and/or features thereof the same as and/or similar to that of the system 4100 as shown and described previously. Similarly, the system 4600 and components thereof may interface with (e.g., contact, couple with, etc.) the foot 4200 and components thereof (e.g., first metatarsal 4202, second metatarsal 4206, medial cuneiform 4206, Lapidus joint 4208, etc.) the same as and/or similarly to that shown with reference to the system 4100. Further, the system 4600 may also be implemented using a surgical methodology that is the same as and/or similar to that of the system 4100, for example the process for implementing a surgical method shown and described herein.

The system 4600, as shown and described herein with reference to FIGS. 55-63, includes a clamp 4602 and may further include one or more additional components, for example one or more surgical guides such as those shown and described herein. Additionally, the system 4600 may also be implemented in performing one or more steps of the method shown and described with reference to FIG. 66. Further, it should be understood that the system 4600 may include one or more components in addition to those shown and described herein (e.g., cutting instruments, saws, scalpels, forceps, retractors, etc.) and accordingly, the method shown and described with reference to FIG. 66 may incorporate the additional components.

FIGS. 55-63 illustrate an exemplary instrument, shown as the clamp 4602, for determining, aligning/realigning, correcting, positioning/repositioning, rotating/derotating, or otherwise manipulating one or more anatomical structures of the foot relative to other anatomical structures of the foot in accordance with the present disclosure. The clamp 4602 is configured to be releasably coupled with structures of a foot that are the same as and/or similar to that shown and described in FIGS. 47-53 (shown as reference numeral 4200) including a first metatarsal (shown as reference numeral 4202) and a second metatarsal (shown as reference numeral 4204) so as to manipulate a Lapidus joint (shown as reference numeral 4208) configured between the first metatarsal 4204 and a medial cuneiform (shown as reference numeral 4206). In some aspects, the clamp 4102 may be implemented on distal portions of the first and second metatarsals 4202, 4204, but may also be implemented elsewhere about the first and second metatarsals 4202, 4204. The clamp (and components thereof) 4602 is configured to facilitate the correction of the IM angle formed between the first extended longitudinal axis of the first metatarsal 4202 and the second extended longitudinal axis of the second metatarsal 4204. Further, the clamp 4602 is configured to facilitate derotation (e.g., rotation opposite that which caused a bunion or other deformity) of the first metatarsal 4202 relative to the second metatarsal 4204. Correction of the IM angle and/or derotation of the first metatarsal 4202 relative to the second metatarsal 4204 may be performed by a physician in a procedure to address a deformity at or near the Lapidus joint (e.g., bunion, arthrodesis of Lapidus joint, etc.).

The clamp 4602 is shown to include a first portion 4604 and a second portion 4606, where the first portion 4604 is releasably coupled with the second portion 4606 by/at a coupling 4608. In some aspects, the coupling 4608 may include a coupling mechanism (e.g., a screw and complimentary bore, etc.) where a first component of the coupling is coupled or integral with the first portion 4604 and a second component complimentary to the first component is coupled with or integral with the second portion 4606. Further, in some aspects, the coupling 4608 may include a portion of a coupling mechanism, where at least a portion of the coupling mechanism is configured to extend at least partially within the second portion 4606. Such a portion of a coupling mechanism may be coupled with or integral with the first portion 4604 and further may extend at least partially into an opening of the second portion 4606. In some aspects, at least a portion of the coupling may include incremental markers (e.g., indicators, measurement markers, etc.) to indicate expansion of the clamp 4602. The coupling 4608 (and any components thereof) may also facilitate motion of the first portion 4604 relative to the second portion 4606, or facilitate motion of the second portion 4606 relative to the first portion 4604. In some aspects, an actuator 4624 (e.g., knob, etc.) positioned substantially along a longitudinal axis of the second portion 4606 may be configured to releasably couple with a portion of the second portion 4606 of the clamp 4602. For example, the actuator 4624 may include a protrusion (e.g., male component with threading, set screw, etc.) that extends longitudinally into and through a bore disposed in the second portion 4606 of the clamp and facilitates releasable coupling at the coupling 4608 with the first portion of the clamp 4604 (e.g., complimentary threads, etc.). In some aspects, the bore may include a coupling mechanism therein (e.g., threading, etc.) configured to releasably couple with a complimentary portion of the actuator 4624 (e.g., the protrusion which may have a coupling mechanism such as threading, etc.). Accordingly, manipulation of the actuator 4624 via a texture surface 4626 (e.g., to facilitate grip, rotation, manipulation, etc.) may translate the second portion 4606 toward or away from the first portion 4604 thus expanding the clamp 4602. In some aspects, the translation may be along a longitudinal axis that is shared by (e.g., common) one or more of the actuator 4624, the protrusion from the actuator 4624, the bore of the second portion 4606, and any complimentary coupling mechanisms of the first portion 4604. Further, the first portion 4604 and the second portion 4606 may be coupled such that one or both portions may be manipulatable about the longitudinal axis (e.g., the shared/common axis).

The second portion 4606 of the clamp 4602 is shown to include a pair of protuberances 4618 extending laterally from the second portion 4606. As shown in FIGS. 55-63, the protuberances 4618 are disposed along the second portion 4606 such that the pair of protuberances are spaced from the actuator 4624 such that the protuberances 4618 are substantially adjacent the coupling 4608 (rather than the alternate position as shown with reference to the system 4100). In some aspects, the actuator 4624 may include at least a portion of a coupling mechanism configured to be complimentary to that of the coupling 4608. For example, the actuator 4624 may include an extension having a threading configured to be received within at least a portion of the coupling 4608. In some aspects, the extension of the actuator 4624 may be configured to releasably couple the second portion 4606 with the first portion 4604 via the coupling 4608. The protuberances 4618 are shown to be substantially hemispherical, although the protuberances 4618 may include alternate geometries in some aspects of the clamp 4602. Each of the protuberances is shown to include a bore 4620 having a substantially cylindrical shape and extending from a top surface through to a bottom surface of the second portion 4606 so as to establish fluid communication therebetween. The bores 4620 may be configured to receive one or more common surgical instruments, for example stabilization wires, k-wires, olive wires, and/or other stabilization elements so as to releasably couple the second portion 4606 with the second metatarsal 4204. Similar to that shown in FIGS. 47-53, the stabilization wires may be placed such that the wires are received by the bores 4620, with the wires inserted into a substantially dorsal portion of the second metatarsal 4204 (and similar to that shown in FIGS. 47-53, positioned nearer the proximal end of the second metatarsal 4204 than the distal end). In some aspects, a physician may elect to place a stabilization wire in one/either of the bores 4620 rather than placing a stabilization wire in each of the bores 4620. When releasably coupled with the second metatarsal 4204 via stabilization wires received through the bores 4620, the second portion 4606 of the clamp 4602 may be configured in a static position should the actuator 4624 be manipulated so as to translate the first portion 4604 toward the second portion 4606 (e.g., the second portion 4606 fixed to the second metatarsal 4204 would be static while manipulation of the actuator 4624 would translate the first portion 4604 toward the second portion 4606 and the second metatarsal 4204). In some aspects, the bores 4620 may be of different sizes (e.g., both larger, both smaller, or one larger/one smaller) and/or geometries (e.g., rectangular, triangular, etc.) so as to accommodate the corresponding geometries of various stabilization elements. Each of the pair of protuberances 4618 are shown to have an equal size, although in some aspects the second portion 4606 of the clamp 4602 may include one, two, or more protuberances the same as or similar to the protuberances 4618 (having equal or unequal sizing) disposed variously along the second portion 4606. Further, in some aspects one or more of the protuberances 4618 may include zero, one, two, or more than two bores arranged various about the protuberances 4618. As shown, each of the bores 4620 are positioned substantially equidistant from the outer edge of the protuberances 4618, although the bores 4620 may be positioned alternately in some aspects of the clamp 4602.

The second portion 4606 of the clamp 4602 is shown to include an extension 4610 configured opposite the second portion 4606 from the coupling 4608 and, as shown in FIGS. 55-63, extending from the second portion 4606 from a point adjacent the actuator 4624 at a substantial orthogonal angle from the second portion 4606. As seen, the extension 4610 has a substantially rectangular cross-sectional geometry, but may have alternate geometries in some aspects of the system 4600. The extension 4610 includes a retention mechanism 4614, shown in FIGS. 47-53 as a pair of protrusions disposed at a distal end of the extension 4610 (e.g., opposite the second portion 4606 adjacent the actuator 4624) defining a cavity 4616 disposed substantially between the pair of protrusions of the retention mechanism 4614. The cavity 4616 of the clamp 4602 may extend further into the extension (e.g., in a direction substantially toward the protuberances 4618) than the extension 4116 of the clamp 4102. As shown, the retention mechanism 4614 is integral to the extension 4610 and has a curvature substantially toward the first portion 4604 of the clamp 4602 (e.g., adjacent a tangent line of the curvature that is substantially perpendicular to the second portion 4606) with the distal-most portions of the retention mechanism 4614 pointing substantially toward the first portion 4604 of the clamp 4602. The cavity 4616 is shown to have a substantially hemi-elliptical geometry, with the hemi-elliptical geometry disposed at proximal-most portion of the cavity 4616. However, in some aspects, the cavity 4616 (as well as the retention mechanism 4614) may have alternate thicknesses/widths, lengths, depths, curvatures, and other geometric properties.

The extension 4610 and the retention mechanism 4614 are configured to at least partially interface with (e.g., engage with, contact, etc.) a flat of the second metatarsal 4204 positioned on a substantially lateral surface of the second metatarsal 4604. The retention mechanism 4614 including the distal-most portion thereof may be configured to contact a substantially inferior portion of the flat on the lateral surface of the second metatarsal 4204. The curvature of the distal-most portion of the retention mechanism 4614 is configured to at least partially surround the second metatarsal 4204 such that the retention mechanism 4614 contacts at least a portion of the plantar surface of the second metatarsal 4204 (and thus engages multiple surfaces of the second metatarsal 4204). The curvature of the retention mechanism 4614 thus accommodates the geometry of the second metatarsal 4204 so as to maximize the engagement of the retention mechanism 4614 with the second metatarsal 4204 should a force be applied to the second metatarsal 4204 via the retention mechanism 4614 (e.g., on a lateral and/or plantar surface of the second metatarsal 4204 and applied in a medial and/or superior direction). Accordingly, the retention mechanism 4614 maximizes the engagement of the extension 4610 of the second portion 4606 of the clamp 4602 with the second metatarsal 4204 so as to reduce/prevent loss of contact/engagement of the clamp 4602 with the second metatarsal 4204 as one or more forces are applied to the second metatarsal 4204 (e.g., manipulation of the actuator 4624 so as to translate one or both of the first portion 4604 and the second portion 4606 toward or away from one another while the extension 4610 and/or the retention mechanism 4614 are in contact with the second metatarsal 4204). In some aspects, the cavity 4616 may be configured to receive (e.g., permit passthrough) of one or more stabilization wires.

The first portion 4604 of the clamp 4602 is shown to have a substantially curved geometry (e.g., convex), with the curvature of the first portion 4604 pointing substantially in the direction of the second portion 4606. The first portion 4604 includes an aperture 4630 disposed substantially on a distal portion 4628 (e.g., distal half) of the first portion 4604. The aperture 4630 as shown in FIGS. 55-63 has an elongated, rounded geometry (e.g., elliptical, oval, etc.) with the aperture 4630 having the same curvature as the first portion 4604. The aperture 4630 is disposed within an inner depression 4633 on the concave surface and an outer depression 4634 on the convex surface of the first portion 4604. The inner depression 4633 and the outer depression 4634 are shown to have similar depth, length, and width dimensions, with the inner depression 4633 and the outer depression 4634 having a greater length and width than the aperture 4630 in the same/a similar geometric shape. Further, the inner depression 4633 and the outer depression 4634 are positioned in approximately the same location on their respective surfaces of the distal portion 4628 of the first portion 4604.

The first portion 4604 is further shown to include an actuator 4636 having a first portion 4638 and a second portion 4640, with the first portion 4638 and the second portion 4640 separated by a connector 4639. The connector 4139 is shown to have a substantially lesser cross-sectional geometry than that of the first portion 4638 or the second portion 4640. In some aspects, the connector 4639 may include an extension having at least a portion of a coupling mechanism (e.g., a threading) configured complimentary to an opening disposed substantially along a longitudinal axis of the first portion 4638. According, such a coupling mechanism may facilitate releasably coupling between the first and second portions 4638, 4640 of the actuator 4636. As shown in FIGS. 55-63, the connector 4639 has a substantially cylindrical geometry with a diameter/lateral dimension slightly lesser than that of the aperture 4630. As shown in FIGS. 55-63, the first portion 4638 is shown to have a substantially cylindrical shape with a diameter slightly lesser than the width of the outer depression 4634. The second portion 4640 is shown to also have a lateral dimension slightly less than the width of the inner depression 4633. As seen in FIGS. 55-63, the actuator 4636 is positioned such that at least a portion of the connector 4639 is disposed within the aperture 4630. Similarly, a distal-most portion of the first portion 4640 is disposed within the outer depression 4634 and the proximal-most portion of the second portion 4640 is disposed within the inner depression 4633. Accordingly, the range of movement of the actuator 4636 is defined by the dimensions of the aperture 4630 (and in some aspects, also the inner depression 4633 and the outer depression 4634). The actuator 4636 may be manipulated through a defined range of motion along the curvature of the first portion 4604, with the bounds of the range of motion defined by the aperture 4630 (and, in some aspects the inner depression 4633 and/or the outer depression 4634). The actuator 4636 also includes a texture 4642 on an outer surface of the first portion 4638 of the actuator 4636, with the texture 4642 configured to facilitate gripping and/or manipulating the actuator 4636 (e.g., through the range of motion, rotation, etc.).

The actuator 4636 includes a bore 4644 extending from the first portion 4638, through the connector 4639 and to the second portion 4640 along a longitudinal axis of the actuator 4636 such that fluid communication is established through the length of the actuator 4636. As shown in FIGS. 55-63, the bore 4644 has a substantially cylindrical geometry configured to receive a stabilization wire (e.g., a k-wire, olive wire, etc.) that may be passed into and through the bore 4644 of the actuator 4636. A first stabilization wire that is the same as or similar to the stabilization wire 4148 shown and described previously may be received by the bore 4644, and inserted into (e.g., releasably coupled with) the first metatarsal 4202 (as shown, a substantially medial portion of the first metatarsal 4202. Accordingly, when the first stabilization wire is placed in the first metatarsal 4202, the actuator 4636 is releasably coupled with the first metatarsal 4202. By manipulating the actuator 4636 within the range of motion defined by the movement of the connector 4639 within the aperture 4630, the first metatarsal 4202 is moved accordingly.

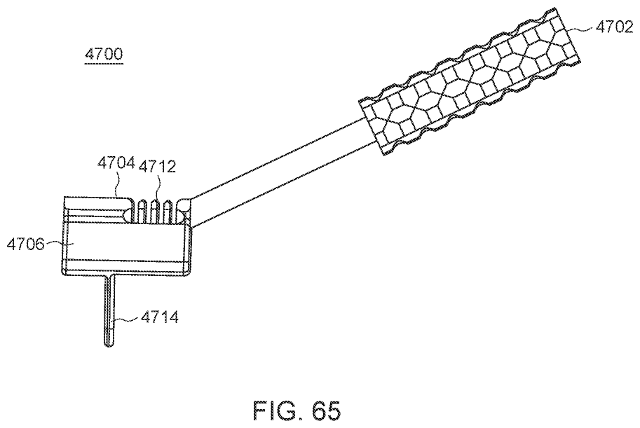

Referring now to FIGS. 64-65, an exemplary embodiment of a surgical guide 4700 is shown. In some aspects, the surgical guide 4700 may be provided as part of a kit or other system along with the system 4600 and/or one or more implants and/or fixation components (as well as various other instruments/components). Further, in some aspects, the surgical guide 4700 may be configured to facilitate one or more cuts made to the first metatarsal 4202 (and may be positioned and/or coupled in the same and/or a similar manner to that shown and described with reference to the cut guide 4300). The surgical guide 4700 includes a handle 4702 extending laterally from a cut guide 4704 of the surgical guide 4700. As shown, an end portion of the handle 4702 includes a tactile finish configured to facilitate the grip of the handle 4702 of the surgical guide 4700. The cut guide 4704 includes slots 4712 positioned in a central portion of the cut guide 4702. As seen in FIGS. 64-65, the slots 4712 have a substantially elongated geometry configured to receive one or more cutting instruments (e.g., reciprocating saw, etc.). In some aspects, the slots 4712 may have alternate geometries and/or dimensions, for example, the slots 4712 may have greater or lesser length and/or width dimensions than those of the slots as shown. The cut guide 4704 is further shown to include an extension 4706 extending laterally from a side of the cut guide 4704 opposite that from which the handle 4702 extends. The extension 4706 includes a bore 4708 substantially centered within the top surface of the extension 4706, with the bore 4708 extending from the top surface of the extension 4706 through to a bottom surface thereof and thus establishing fluid communication between the top and bottom surfaces of the extension 4706. The bore 4708 is configured to receive a stabilization wire (which may be the same as and/or similar to the stabilization wires shown and described previously herein) therethrough such that the stabilization wire may releasably couple the surgical guide 4700 with the proximal portion of the first metatarsal 4202. In some aspects, the surgical guide 4700 and the extension 4706 thereof may include a plurality of bores 4708, for example a pair of bores 4708. In some aspects, one or more stabilization wires may be received therethrough the bore/bores 4708 to releasably couple the surgical guide 4700 with the first metatarsal 4202 at one or more points. Further, in some aspects the surgical guide 4700 includes a protrusion 4714 (e.g., a paddle, etc.,) configured to be inserted into the joint space of the Lapidus joint 4208 (e.g., between the first metatarsal 4202 and the medial cuneiform 4206).

Referring now to FIG. 66, a process or method 6000 for performing a procedure of the Lapidus joint is shown, according to an exemplary embodiment. The process 6000 and the steps thereof as shown in FIG. 66 may be performed by implementing one or more of the components as shown and described herein with reference to FIGS. 39-65. Additionally, it should be noted that one or more of the steps of the process 6000 may be performed in an alternate order (e.g., out of order), repeated, skipped, or otherwise modified. It should also be noted that the steps of the process 6000 may not be limiting, which is to say that additional steps may be added to the process 6000 in performing a surgical procedure on the Lapidus joint. The steps of the process 6000 as described herein may include references to one or more components of the systems 4100, 4600 as shown and described previously here. In some aspects, one or more of the steps of the process 6000 may include a component of one of the systems 4100, 4600, and may be noted as such (e.g., inclusion of components in the format of “systems 4100, 4600” does not require both components or both systems to be implemented in order for the step to be completed).

The process 6000 is shown to include a step 6002 coupling a portion of a device with a second metatarsal with at least one stabilization wire, according to some aspects. In some aspects, the step 6002 may include implementing one or more stabilization wires such as those shown and described previously herein. The step 6002 may correspond to coupling the second portion 4106, 4606 with the second metatarsal 4204 as shown and described previously herein.

The process 6000 is shown to include a step 6004 coupling a portion of the device with the first metatarsal, according to some aspects. In some aspects, the step 6004 may include implementing one or more stabilization wires such as those shown and described previously herein. The step 6004 may include coupling the first portion 4104, 4604 with the first metatarsal 4202 as shown and described previously herein.

The process 6000 is shown to include a step 6006 coupling a surgical guide adjacent the Lapidus joint, according to some aspects. In some aspects, the step 6006 may include coupling a surgical guide the same as or similar to the surgical guide 4300 as shown and described previously. The surgical guide of the step 6006 may be coupled with at least the first metatarsal 4202 at a substantially proximal portion thereof using one or more stabilization wires as described previously herein.

The process 6000 is shown to include a step 6008 preparing the proximal portion of the first metatarsal, according to some aspects. The step 6008 may include implementing one or more cutting instruments (as well as other possible instruments) in order to prepare the proximal portion of the first metatarsal 4202. In performing the step 6008, one or more of the aforementioned cutting instruments may be inserted into/through one or more slots 4312, as shown with reference to the surgical guide 4300.

The process 6000 is shown to include a step 6010 manipulating an actuator to decrease the distance between the first and second metatarsals by decreasing the distance between the first and second portions of the device, according to some aspects. In some aspects the actuator manipulated in the step 6010 may be the actuator 4124, 4624 as shown and described with reference to FIGS. 39-54, where the actuator 4124, 4624 may be actuated (e.g., rotated) so as to decrease the distance between the first portion 4104, 4604 and the second portion 4106, 4606 of the clamp 4102, 4602. Accordingly, by decreasing such distance with the first and second portions 4104, 4604, 4106, 4606 coupled with the first and second metatarsals 4202, 4204, respectively, the distance between the first and second metatarsals is decreased.

The process 6000 is shown to include a step 6012 manipulating an actuator to rotate the first metatarsal, according to some aspects. In some aspects, the actuator manipulated in the step 6012 may be the actuator 4136, 4636 as shown and described with reference to FIGS. 39-65, where the actuator 4136, 4636 may be translated (e.g., manipulated) about/through a defined range of motion. Such manipulation of the actuator 4136, 4636 may rotate the first metatarsal 4202 in a substantially counterclockwise direction when viewed from an anterior perspective (as applied to a right foot of a patient).

The process 6000 is shown to include a step 6014 coupling a surgical guide adjacent the Lapidus joint, according to some aspects. In some aspects, the surgical guide of the step 6014 may be the same as and/or similar to the surgical guide 4400, 4700 as shown and described with reference to FIGS. 52 and 64-65. The step 6014 may further include manipulating one or more components of the surgical guide 4400, 4700 (such as those shown and described previously to be manipulatable, pivotable, etc.) and subsequently coupling one or more portions/components of the surgical guide 4400, 4700 with the distal portion of the medial cuneiform 4206 using one or more stabilization wires. In some aspects, the step 6014 may include positioning and/or manipulating the surgical guide 4400, 4700 such that inserting one or more cutting instruments into/through the cut slot 4412, 4712 may prepare the distal portion of the medial cuneiform 4206 to have a surface substantially parallel (e.g., parallel planes) to that of the proximal surface of the first metatarsal 4202.

The process 6000 is shown to include a step 6016 preparing the distal portion of the medial cuneiform, according to some aspects. In some aspects, the step 6016 may include preparing the distal-most surface of the medial cuneiform 4206 such that it is parallel with the corresponding proximal surface of the first metatarsal 4202 (e.g., so as to promote maximum surface area contact between the distal surface of the medial cuneiform 4206 and the proximal surface of the first metatarsal 4202). The step 6016 may include implementing one or more cutting instruments (as well as other possible instruments) in order to prepare the distal portion of the medial cuneiform 4206. In performing the step 6016, one or more of the aforementioned cutting instruments may be inserted into/through one or more slots 4412, 4712 as shown with reference to the surgical guide 4400.

The process 6000 is shown to include a step 6018 applying a fusion device across the Lapidus joint. In some aspects, the fusion device of the step 6018 may include an intramedullary nail and/or one or more plating components (including but not limited to those incorporated by reference herein). The fusion device may be coupled with the first metatarsal 4202, the medial cuneiform 4206, and in some aspects, may further be coupled with one or more musculoskeletal structures near the Lapidus joint 4208 using one or more fastening means (e.g., screw, fastener, etc.).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. A clamp, comprising:
an actuation portion; and
an engagement portion coupled with the actuation portion by a coupling portion, the engagement portion comprising:
a first arm comprising a first engagement element arranged at a distal end of the first arm; and
a second arm comprising a second engagement element arranged at a distal end of the second arm.

2. The clamp according to claim 1, wherein the first engagement element is different from the second engagement element.

3. The clamp according to claim 2, wherein the second engagement element is integral with the second arm.

4. The clamp according to claim 3, wherein the second engagement element comprises a tapered geometry in the vertical direction.

5. The clamp according to claim 4, wherein the second engagement element comprises a flat disposed on a medial surface thereof, wherein the flat comprises a textured surface.

6. The clamp according to claim 5, wherein the textured surface is configured to contact at least a portion of a first metatarsal such that the second engagement element maintains contact with the first metatarsal.

7. The clamp according to claim 1, wherein the first arm comprises an aperture positioned at a distal end, wherein the aperture is configured to rotatably couple with the first engagement element.

8. The clamp according to claim 7, wherein the first engagement element comprises a retention portion and an actuator.

9. The clamp according to claim 8, wherein the retention portion comprises a post, at least a portion of which is configured to be received by the aperture of the first arm so as to facilitate rotatable and releasable coupling between the first engagement element and the first arm.

10. The clamp according to claim 8, wherein the actuator comprises a central opening configured to receive at least a portion of the post such that the actuator is disposed below the aperture of the first arm and above the retention portion.

11. The clamp according to claim 9, wherein the retention portion further comprises a first retention element and a second retention element extending from the post.

12. The clamp according to claim 11, wherein manipulation of the actuator about a threading disposed on a surface of the retention portion adjusts a distance between the first retention element and the second retention element.

13. The clamp according to claim 11, wherein the first retention element and the second retention element each comprise a textured surface configured to interface with a second metatarsal.

14. The clamp according to claim 8, wherein the retention portion is rotatably coupled with the aperture of the first arm such that the retention portion comprises a 360-degree range of motion.

15. The clamp according to claim 1, wherein the first arm has a different geometry from the second arm.

16. The clamp according to claim 1, wherein the first engagement element has a depth different than that of the second engagement element.

17. A surgical system, comprising:
a clamp, comprising:
an actuation portion; and
an engagement portion coupled with the actuation portion by a coupling portion disposed between the engagement portion and the actuation portion, the engagement portion comprising:
a first arm comprising a first engagement element arranged at a distal end of the first arm, wherein the first engagement element is rotatably and releasably coupled with the first arm and comprises an actuator configured to adjust a distance between a first and second retention element such that the first and second retention elements each engage at least a portion of a second bone;
a second arm comprising a second engagement element arranged at a distal end of the second arm, wherein the second engagement is element integral with the second arm and configured to contact a surface of a first bone; and
a fixation device.

18. The surgical system according to claim 17, wherein manipulating the actuation portion increases or decreases the distance between the first and second engagement elements.

19. The surgical system according to claim 17, wherein the first engagement element comprises a retention portion comprising a post and a threading, wherein the post is configured to releasably couple with the first arm and the threading is configured to releasably couple with the actuator.

20. A surgical method, comprising:
aligning a retention element comprising a post and a pair of extensions positioned opposite the post such that a first bone is disposed at least partially between each of the pair of extensions;
releasably coupling a surgical clamp with the post of the retention element such that the post is received by a portion of a first arm of the surgical clamp;
manipulating an actuator such that each of the pair of extensions contact and releasably couple with at least a portion of the first bone;
manipulating the surgical clamp such that an engagement element disposed on a second arm of the surgical clamp contacts a surface of a second bone;
coupling a first stabilization wire with the second bone;
reducing an intramedullary angle between the first bone and the second bone;
derotating the second bone by manipulating the first stabilization wire;
inserting a second stabilization wire through a through hole of the second arm and into the second bone;
making one or more cuts to one or more bones; and
applying a fusion device across the first bone and a third bone.

21. A surgical system, comprising
a clamp, comprising:
a first portion comprising a first actuator configured to interface with a first metatarsal of a patient, wherein a range of movement of the first actuator is defined by an aperture;
a second portion, comprising an extension having a retention mechanism configured to interface with a second metatarsal of the patient;
wherein the second portion is releasably coupled with the first portion and the first and second portions are translatable relative to one another via a second actuator; and
a plurality of stabilization wires configured to be received by at least one of the first portion and the second portion.

22. The surgical system according to claim 21, wherein the first actuator comprises a first bore disposed about a longitudinal axis of the actuator.

23. The surgical system according to claim 22, wherein the first bore is configured to receive one of the plurality of stabilization wires into and through the bore such that the stabilization wire releasably couples the first actuator with the first metatarsal.

24. The surgical system according to claim 23, wherein manipulation of the first actuator within the aperture applies a rotational force to the first metatarsal via at least one of the plurality of stabilization wire so as to manipulate the first metatarsal from a first position to a second position.

25. The surgical system according to claim 24, wherein manipulation of the first actuator within the aperture moves at least one of the plurality of stabilization wires from a first position forming a first angle with the transverse plane to a second position forming a second position forming a second angle with the transverse plane, wherein the first angle is different than the second angle.

26. The surgical system according to claim 25, wherein the first angle is greater than the second angle.

27. The surgical system according to claim 21, wherein the retention mechanism is disposed at a distal end of the extension and comprises a pair of curved protrusions defining a recess therebetween.

28. The surgical system according to claim 27, wherein the protrusions are curved in the direction of the first portion of the clamp.

29. The surgical system according to claim 21, wherein the second actuator is disposed at an end of the second portion opposite the first portion.

30. The surgical system according to claim 21, wherein the second portion comprises a bore extending through the second portion along a longitudinal axis thereof.

31. The surgical system according to claim 21, wherein the second actuator comprises a protrusion having a first coupling mechanism, the protrusion configured to be received by and extend through the bore, wherein the protrusion is configured to releasably couple with the second portion via the bore.

32. The surgical system according to claim 29, wherein the first portion comprises a recess comprising a second coupling mechanism, the recess configured to receive and releasably couple with a portion of the protrusion of the second actuator.

33. The surgical system according to claim 32, wherein the recess of the first portion and the bore of the second portion share a common axis of the protrusion when both are releasably coupled with the protrusion of the second actuator.

34. The surgical system according to claim 21, wherein manipulation of the second actuator translates the first portion relative to the second portion about the shared axis.

35. The surgical system according to claim 21, wherein the first and second actuators each comprise a texture disposed on one or more outer surfaces thereof.

36. The surgical system according to claim 31, wherein the second portion comprises one or more protuberances each comprising an aperture configured to receive one of the plurality of stabilization wires.

37. A surgical clamp, comprising
a first portion having a substantially curved geometry, the first portion comprising:
a recess positioned at a proximal end of the first portion;
an elongated aperture positioned at a distal end of the first portion; and
a first actuator disposed at least partially within the elongated aperture such that the elongated aperture defines a range of movement of the first actuator, wherein the first actuator comprises a bore extending therethrough along a longitudinal axis thereof and configured to receive a stabilization wire;
a second portion, comprising:
an extension comprising a pair of protrusions at a distal end thereof;
a bore extending along a longitudinal axis of the second portion; and
a second actuator comprising a coupling mechanism configured to be received through and releasably couple with the bore, wherein the coupling mechanism is further configured to be received by releasably and couple with the recess such that manipulation of the second actuator translates the first portion and the second portion about the longitudinal axis of the second portion.

38. The surgical clamp according to claim 37, wherein the first portion comprises at least one bore configured to receive a stabilization wire.

39. The surgical clamp according to claim 37, wherein the second portion comprises at least aperture configured to receive a stabilization wire.

40. A method of performing Lapidus joint procedure, comprising:
coupling a first portion of a device with a second metatarsal with a first and second stabilization wire;
coupling a second portion of the device with the first metatarsal with a third stabilization wire, wherein the third stabilization wire is received by a first actuator of the second portion;
manipulating a second actuator disposed on the first portion of the device so as to decrease the distance between the first and second metatarsals;
manipulating the first actuator so as rotate the first metatarsal;
coupling a surgical guide adjacent the Lapidus joint with a fourth stabilization wire;
preparing a distal portion of a medial cuneiform and a proximal portion of the first metatarsal for fusion such that the distal portion of the medial cuneiform comprises a surface that is substantially parallel to a proximal surface of the first metatarsal; and
applying a fusion device across the Lapidus joint.
